(19) United States
(12) Patent Application Publication
(10) Pub. No.: US 2013/0041236 A1
(43) Pub. Date: Feb. 14, 2013

(54) SAMPLE ANALYSIS SYSTEM AND METHOD OF USE

(71) Applicants: Michael J. Pugia, Granger, IN (US); Gregory Paul Panebianco, Waltham, MA (US); Henry Lu, Bloomington, IN (US)

(72) Inventors: Michael J. Pugia, Granger, IN (US); Gregory Paul Panebianco, Waltham, MA (US); Henry Lu, Bloomington, IN (US)

(73) Assignee: SIEMENS HEALTHCARE DIAGNOSTICS INC., Tarrytown, NY (US)

(21) Appl. No.: 13/643,633

(22) PCT Filed: Apr. 22, 2011
(86) PCT No.: PCT/US11/33556

§ 371 Date: Oct. 26, 2012

Related U.S. Application Data
(60) Provisional application No. 61/328,794, filed on Apr. 28, 2010.

(51) Int. Cl. A61B 5/20; G01N 33/53; G01N 33/48; B23P 17/04

(52) U.S. Cl. 600/309; 29/428; 422/430; 422/68.1; 435/287.1; 435/289.1

(57) ABSTRACT

A sample collection device having a sample container and microfluidic device having one or more microfluidic circuits, the system for analyzing biological samples. The microfluidic device has a sample inlet port, a microconduit in communication with the inlet port and with reaction chamber. The reaction chamber is connected to an air vent via another microconduit. Air may be vented from the microfluidic circuit via the air vent of the microfluidic circuit via an air vent in the sample container.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

1. Field of the Presently Disclosed and Claimed Inventive Concept(s)

The presently disclosed and claimed inventive concept(s) relates to a system for collecting and analyzing patient samples. In particular, the presently disclosed and claimed inventive concept(s) provides an improved sample analysis system and method that greatly reduces the labor and the likelihood of errors involved in collecting and analyzing patient samples. The presently disclosed and claimed inventive concept(s) also relates to sample analysis systems which include microfluidic devices, particularly those that are used for analysis of biological samples.

2. Background of the Presently Disclosed and Claimed Inventive Concept(s)

Various types of analytical tests related to patient diagnosis and therapy can be performed by analysis of a liquid sample taken from a patient's infections, bodily fluids or abscesses. These assays are typically conducted with automated clinical analyzers onto which tubes or vials containing patient samples have been loaded. The analyzer extracts liquid sample from the vial and combines the sample with various reagents in special reaction cuvettes or tubes. Usually the sample-reagent solution is incubated or otherwise processed before being analyzed. Analytical measurements are often performed using a beam of interrogating radiation interacting with the sample-reagent combination to generate turbidimetric, fluorometric, absorption readings or the like. The readings allow determination of end-point or rate values from which an amount of analyte related to the health of the patient may be determined using well-known calibration techniques.

Patient samples are known to be provided to such analyzers in a large number of different types of tubes: 13 mm and 16 mm diameter tubes are popular as are “small sample” tubes, sometimes called sample cups, and tubes are also used having varying heights. After being placed on the analyzer, a predetermined, known portion of the original sample is aspirated from the tube and analytical tests conducted thereon. Sample racks with features for accommodating different types of tubes may be found in U.S. Pat. Nos. 5,687,849; 5,378,433; and 4,944,942; an adapter for accommodating different types of tubes may be found in U.S. Pat. No. 5,985,219; and a micro-sample cup rack adapter is described in U.S. Pat. No. 7,569,190, the entire content of each of which is hereby expressly incorporated by reference in their entirety.

With respect to the analysis instrument market, it is common for companies to provide a family of different instruments for different segments of the market. For example, the current urinalysis instrument market can be divided into three categories: one focused on the small doctor's clinics, one focused on the larger clinics/small hospitals, and one focused on the large hospitals and clinical laboratories. Exemplary instruments for the small doctor's clinics; larger clinics/small hospitals; and large hospitals and clinical laboratories are sold under the tradenames Clinitek Status; Clinitek Advantus and Clinitek Atlas. In particular, to fulfill the need for the entire market, a company would need between two to four distinct instrument offerings each with its own production line, development phase, etc. This increases the costs associated with the development and manufacture of the family of analysis instruments.

The use of conventional analysis instruments is also labor intensive. In particular, the conventional urinalysis instruments including the automated machines still require a significant amount of manual labor to operate. On the small and medium scale level instruments, a customer would require manual labor to collect the urine, transfer the urine to a test tube, manually test the urine and tabulate the results. On the large scale automated instrument market, the hospital would still need to manually collect the urine sample, transfer the samples into test tubes, label the individual test tubes, store the samples for periodic testing, and tabulate the sample results.

Microfluidic devices are known in the art and intended to be used for rapid analysis of samples, thus avoiding the delay inherent in sending biological samples to a central laboratory. Such devices are intended to accept very small samples of blood, urine, and other biological samples. The samples are brought into contact with reagents capable of indicating the presence and quantity of analytes found in the sample.

Many devices have been suggested for carrying out analysis near the patient. Microfluidic devices have many advantages over the use of dry reagent strips for testing in the near-patient environment. In general, such devices use only small sample volumes, typically 0.1 to 200 μL. With the development of microfluidic devices the samples have become smaller, which is a desirable feature of their use. However, smaller samples introduce difficult problems. In microfluidic devices, small sample volumes, typically about 0.1 to 20 μL, are brought into contact with one or more wells where the samples are prepared for later analysis or are immediately reacted to indicate the presence (or absence) of an analyte. As the sample is moved into a well or chamber for immediate or later reaction, it is important that the liquid is uniformly distributed such that all the air in the well is expelled, since air will adversely affect the movement of liquid and the analytical results. Also, there are other problems associated with the initial introduction of the sample to the microfluidic device.

For example, the interaction of the sample with the walls of the microfluidic device is critical to its performance. The sample must be moved in the desired amounts through the capillaries and chambers and must contact dry reagents therein uniformly, while purging the air that initially filled the spaces in the device. The present invention is concerned, for example, with solving problems related to this process.

At first, the inlet port of such devices contains air, which must be expelled. A small amount of liquid must be deposited under conditions which force air out, but leave the sample in the inlet port and not on the surface of the device because specimens on the surface may cause carry-over and contamination between different samples. Air in the port may cause under-filling and, consequently, under estimation of the analytical results. Air bubbles in the inlet port or the receiving inlet chamber might interfere with the further liquid handling, especially if lateral capillary flow is used for further flow propulsion. One solution which has been used is to seal the inlet port to a pipette containing the sample liquid so that a plunger in the pipette can apply pressure to the inlet port. The flow through a capillary extending from the inlet port to the first well must prevent air bubbles from forming in the capillary or in the entry to the first well. As the capillary enters the first well, the liquid should be distributed evenly as the passageway widens into the well. Here also, the movement of the liquid must be controlled so that air is moved ahead of the liquid and expelled through a vent passage. The goal is to force all the air in the well to exit via the vent as it is replaced with the liquid sample. If the vent passage is blocked by liquid before all of the well air has escaped, air bubbles will form in the well and reduce the accuracy of the test.

While the sample may be directed immediately to a well containing reagents, instead it may be sent first to a metering well used to define the amount of the sample which later will be sent to other wells for preparation of the sample for subsequent contact with reagents. It is important that the metering is completely filled with a liquid sample rather than air. If the well is under-filled due to the presence of air bubbles then the measurements are affected because less liquid is available for the analysis. If the well is over-filled, excess liquid may enter the downstream micro fluidic circuit and interfere with the processing of the correct sample volume. Consequently, an overflow well may be provided to accommodate liquid in excess of the sample to be assayed. Since precision in metering a sample requires that all the air originally in the well be expelled, the method used to introduce a sample liquid into a well that defines the volume to be assayed should prevent trapping of air.

In particular, it is important that correct amounts of sample fluid be able to move accurately within the microfluidic device. Previous systems have often suffered from the inability to induce and cause accurate fluid flows within the device.

These issues have become even more important because diagnostic systems for Point of Care (POC) testing are continuously becoming smaller, less costly, and capable of performing more than one type of testing. In one example, a bench top urinalysis instrument must read blood immunoassays in a chromatography cassette along with a urinalysis strip. Rising healthcare costs are causing diagnostics suppliers to seek process improvements which reduce the cost to deliver high quality clinical information. One means to reduce costs is to eliminate steps and components used in the process. Blood collection tubes and urine cup processes account for substantial labor and materials in the total cost of delivering a diagnostic result. For example, a customer may be required to obtain the sample in the tube or cup, and transport it to the point of testing where the clinician tests the sample with a reagent.

Technologies allowing miniaturization have enabled designers to increase the types of testing per given space and to decrease the manufacturing cost per result. For example, the following four miniaturization technologies have been previously developed:

First, molding of μm fluidic (microfluidic) patterns into plastics allows miniaturization of the reagent amounts and produces smaller and low cost disposables for diagnostics. These microfluidic patterns allow liquid and dry reagents to be combined to produce lab quality results conveniently in a POC testing setting. Microfluidics also reduces the amounts of expensive reagent biochemicals used. This is important as biochemicals are essential for use in affinity capture; a fluidic process of passing liquid through a binding area to amplify binding of the biochemical to the analyte of interest. This amount of analyte bound is measured by use of labels, such as enzyme labels, to further amplify and produce a detectable signal.

Second, miniaturized optical designs (micro-optics or MORH) using μm-sized LEDs, μm-sized photodiodes and light guides are capable of reading mm-sized reagent areas in the microfluidic disposables. These micro-optic designs allow smaller and lower cost instruments.

Third, delivery of miniaturized volumes of liquid reagents (pL to μL) has been achieved using μm-sized nozzles. These nozzles are opened on demand by piezo-ceramic electronics, for example, allowing μsec timing of liquid additions. Since these nozzles release droplets from a distance, the liquid reagent can be separated and not directly contact the microfluidic disposable. This improves storage stability and allows liquids to be held in reservoirs used many times over longer periods.

Fourth, micro-volumes of sample are a sensitivity and detection challenge. A minimum sensitivity of 10⁻¹² to 10⁻¹³ M is needed for immunoassay and nucleic acid analysis. High sensitivity electromechanical analyzers miniaturized to small areas (e.g., 1-10 mm²) must be capable of measuring small volumes (e.g., 0.1-20 μL). Nanometer electrode patterns are effective but cost effective fabrication and scale up of are required. Fabrication and scale up of detection can be achieved with Complementary metal-oxide-semiconductor (CMOS) technology for example.

However, there are problems in most effectively and efficiently combining all of these elements in a simple system.

The presently claimed and disclosed inventive concept(s) has been developed to overcome the problems discussed above and to provide accurate and repeatable results.

### SUMMARY OF THE DISCLOSURE

The presently claimed and disclosed inventive concept(s) relates to microfluidic analysis devices adapted to treat small samples of, for example, 0.1 to 20 μL, thereby making possible accurate and repeatable assays of the analytes of interest in such samples. The devices have one or more microfluidic analysis units each comprising a microfluidic circuit having an entry port which provides access for small samples of fluid and for transfer of the samples into a sample chamber while purging air from the system without trapping air bubbles therein. Uniform distribution of the fluid sample and venting of air may be facilitated by various structures such as, but not limited to, chambers, microconduits, and air vents.

The microfluidic device of the presently claimed and disclosed inventive concept(s) may include one or more overflow chambers, reaction chambers, microconduits with capillary stops, and air vents. The capillary stops direct the fluid flow in a preferred direction.

In one aspect, the presently claimed and disclosed inventive concept(s) includes a method of supplying a liquid sample to a microfluidic analysis device in which liquid is introduced to a sample inlet port, where from it flows through a capillary passageway (microconduit) by capillary forces into a reaction chamber, for example via a sample chamber, where the liquid sample is exposed to a reaction substrate while completely purging air from the chamber(s) and microconduits through at least one air vent. In preferred embodiments, capillary stops which comprise narrow passageways between the chambers and air vent cause the fluid to flow unidirectionally toward the reaction chamber. Excess fluid may flow into an overflow chamber, where such overflow chamber is present.

In one aspect, the presently claimed and disclosed inventive concept(s) includes a kit for a sample collection device comprising a sample container and a microfluidic device. The sample container has a sidewall, an inner space, a sample outlet and an air conduit. The microfluidic device is attachable to the sample container and has at least one microfluidic circuit, wherein when the microfluidic device is attached to the sample container, the microfluidic circuit is placed in fluid communication with the sample outlet and the air conduit of the sample container, and the microfluidic circuit having a reaction chamber for receiving a fluid sample from the sample container.

In one aspect, the presently claimed and disclosed inventive concept(s) includes a kit for analyzing biological samples comprising a sample collection device and a portable reader. The sample collection device includes a container and a reagent device. The container defines a collection space adapted to collect and retain a sample directly from a patient. The container has a bottom. The reagent device is located adjacent to the bottom of the container and is in communication with the collection space to receive a portion of the sample. The portable reader comprises (1) a computer readable medium storing a code identifying at least one of a patient and a sample, (2) an analyzer and (3) a signal transceiver. The portable reader is configured to mate with the container of the sample collection device for positioning the analyzer below the bottom of the container wherein when the portable reader is mated with the container and a read cycle is initiated the analyzer analyzes the reagent device to generate data indicative of the analysis of the reagent device and the signal transceiver outputs the code and the data indicative of the reagent device.

In another aspect, the presently claimed and disclosed inventive concept(s) includes a portable reader for automatically analyzing a sample collected from a patient with a sample collection device having a container defining a collection space of at least 75 mL and a reagent device positioned adjacent to a bottom of the container. The portable reader comprises a computer readable medium, an analyzer and a signal transceiver. The computer readable medium is initialized with a code identifying at least one of a patient and a sample. The analyzer is adapted to analyze the reagent device from a position beneath the bottom of the container. The signal transceiver is adapted to output the code and data indicative of the analysis of the reagent device.

In yet another aspect, the presently claimed and disclosed inventive concept(s) includes a kit for performing urinalysis, comprising a sample collection device, a portable reader and a host system. The sample collection device includes a container, and a reagent device. The container defines a collection space adapted to collect and retain urine directly from a patient. The reagent device is in communication with the collection space to receive a portion of the urine. The portable reader comprises an analyzer adapted to optically read the reagent device from a position below the container. The portable reader includes a signal transceiver adapted to output (1) a unique code indicative of at least one of a patient and a sample, and (2) raw data indicative of the analysis of the reagent device. The host system is adapted to execute a medical database and store the unique code and readable results into the medical database with the readable results indicative of the analysis of the reagent device.

In yet another aspect, the presently claimed and disclosed inventive concept(s) includes a sample collection device comprising a container and a reagent device. The container has a bottom, and defines a collection space adapted to collect and retain a sample directly from a patient. The container also defines a reaction chamber adjacent to the bottom with the collection space and the reaction chamber having a volumetric ratio of at least 100 to 1. The container is configured to establish fluid communication between the collection space and the reaction chamber. The reagent device is positioned in the reaction chamber and extends across a portion of the bottom of the container to be optically readable from a position beneath the container.

### BRIEF DESCRIPTION OF THE DRAWINGS

To assist those of ordinary skill in the relevant art in making and using the subject matter hereof, reference is made to the appended drawings, which are not intended to be drawn to scale, and in which like reference numerals are intended to refer to similar elements for consistency. For purposes of clarity, not every component may be labeled in every drawing.

FIG. 1 is a schematic view of a sample analysis system constructed in accordance with one embodiment of the presently disclosed and claimed inventive concept(s).

FIG. 2 is another schematic view of the sample analysis system of FIG. 1 showing a block diagram of an exemplary portable reader.

FIG. 3 is a flow diagram of logic stored on a computer readable medium, that when executed by one or more processor causes the one or more processor to execute the steps of the process.

FIG. 4 is a perspective view of an exemplary portable reader constructed in accordance with the presently disclosed and claimed inventive concept(s).

FIG. 5 is a top plan view of the portable reader of FIG. 4.

FIG. 6 is a bottom plan view of the portable reader of FIG. 5.

FIG. 7 is a perspective view of a sample collection device constructed in accordance with the presently disclosed and claimed inventive concept(s) with the sample collection device constructed of a transparent material.

FIG. 8 is a bottom plan view of the sample collection device of FIG. 7 illustrating a transparent bottom of the sample collection device.

FIG. 9 is a fragmental, cross-sectional view of the sample collection device of FIGS. 7 and 8 showing a reagent device encapsulated at a bottom of the sample collection device.

FIG. 10 is a side elevation view of an exemplary embodiment of the reagent device constructed in accordance with the presently disclosed and claimed inventive concept(s).

FIG. 11 is a perspective view of the portable reader of FIGS. 4-6 mated with the sample collection device of FIGS. 7-9.

FIG. 11a is fragmental, cross-sectional view of the portable reader mated with the sample collection device as depicted in FIG. 11 showing an analyzer positioned in a bottom of the portable reader.

FIG. 11a is fragmental, cross-sectional view of alternative versions of a portable reader mated with a sample collection device showing an analyzer positioned in a sidewall of the portable reader.

FIG. 12 is a perspective view of a plurality of the portable readers positioned on a base station in accordance with the presently disclosed and claimed inventive concept(s).

FIG. 13 is a block diagram of an exemplary embodiment of the base station of FIG. 12.

FIG. 14 is a schematic representation of a microfluidic device constructed in accordance with the present invention.

FIG. 15A is a cross-sectional view of the microfluidic device of FIG. 14 taken through line 15A-15A.

FIG. 15B is a cross-sectional view of the microfluidic device of FIG. 14 taken through line 15B-15B.

FIG. 15C is a cross-sectional view of the microfluidic device of FIG. 14 taken through line 15C-15C.

FIG. 16 is a schematic representation of a microfluidic device constructed in accordance with the present invention.

FIG. 17A is a cross-sectional view of the microfluidic device of FIG. 16 taken through line 17A-17A.

FIG. 17B is a cross-sectional view of the microfluidic device of FIG. 16 taken through line 17B-17B.

FIG. 17C is a cross-sectional view of the microfluidic device of FIG. 16 taken through line 17C-17C.

FIG. 18 is a schematic representation of a microfluidic device constructed in accordance with the present invention.

FIG. 19A is a cross-sectional view of the microfluidic device of FIG. 18 taken through line 19A-19A.

FIG. 19B is a cross-sectional view of the microfluidic device of FIG. 18 taken through line 19B-19B.

FIG. 19C is a cross-sectional view of the microfluidic device of FIG. 18 taken through line 19C-19C.

FIG. 19D is a cross-sectional view of the microfluidic device of FIG. 18 taken through line 19D-19D.

FIG. 20 is a schematic representation of a reaction chamber of a microfluidic device of the presently claimed and disclosed inventive concept(s) having a reagent substrate therein.

FIG. 21A is a cross-sectional view of FIG. 20 taken through line 21-21 show the reagent substrate therein in a preferred configuration.

FIG. 21B is a cross-sectional view taken through line 21-21 of FIG. 20 showing the reagent substrate therein in an alternate configuration.

FIG. 21C is a cross-sectional view taken through line 21-21 of FIG. 20 showing the reagent substrate therein in another alternate configuration.

FIG. 22 is a schematic representation of a reaction chamber of a microfluidic device of the presently claimed and disclosed inventive concept(s) having a plurality of reaction wells disposed therein each containing a reagent or reagent substrate therein.

FIG. 23 is a schematic representation of a reaction chamber of a microfluidic device of the presently claimed and disclosed inventive concept(s) having a plurality of separate reagent substrates positioned therein.

FIG. 24 is a schematic representation of an alternate embodiment of a reaction chamber of the presently claimed and disclosed inventive concept(s) which has a pair of separate chambers connected by a microconduit.

FIG. 25 is a schematic representation of an alternative embodiment of a microfluidic device constructed in accordance with the presently claimed and disclosed inventive concept(s), and comprising a plurality of microfluidic units.

FIG. 26 is a schematic representation of an alternative embodiment of a microfluidic device constructed in accordance with the presently claimed and disclosed inventive concept(s), and comprising a plurality of microfluidic units.

FIG. 27 is a cross-sectional view of a sample collection device having a microfluidic device connected to a base thereof.

FIG. 28 is a cross-sectional view of the sample collection device having a urine sample contained therein.

FIG. 29 is a cross-sectional view of a sample collection device having a closure seal on the base thereof and a microfluidic device of the presently claimed and disclosed inventive concept(s).

FIG. 30 is a cross-sectional view of a sample collection device and a microfluidic device of the presently claimed and disclosed inventive concept(s) which has a puncturable sealing and/or adhesive layer disposed over an upper surface thereof.

FIG. 31 is a perspective view of a sample collection device having a microfluidic device of the presently claimed and disclosed inventive concept(s) which is movably attached to a base thereof.

FIG. 32 is a cross-sectional view of FIG. 31 taken through line 32-32.

### DETAILED DESCRIPTION OF THE INVENTION

The description herein of several embodiments describes non-limiting examples that further illustrate the presently claimed and disclosed inventive concept(s).

In the following detailed description, numerous specific details are set forth in order to provide a more thorough understanding of the disclosure. However, it will be apparent to a person having ordinary skill in the art that the presently claimed and disclosed inventive concept(s) may be practiced without these specific details. In other instances, features which are well known to persons of ordinary skill in the art have not been described in detail to avoid complication unnecessarily the description.

Therefore, unless defined otherwise, all technical and scientific terms used herein have the same meanings as commonly understood by one skilled in the art to which the presently claimed and disclosed inventive concept(s) pertains. For example, the term “plurality” refers to “two or more.” The singular forms “a,” “an,” and “the” include plural referents unless the context clearly indicates otherwise. Thus, for example, reference to “a reaction chamber” refers to 1 or more, 2 or more, 3 or more, 4 or more or greater numbers of reaction chambers. The term “about”, where used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

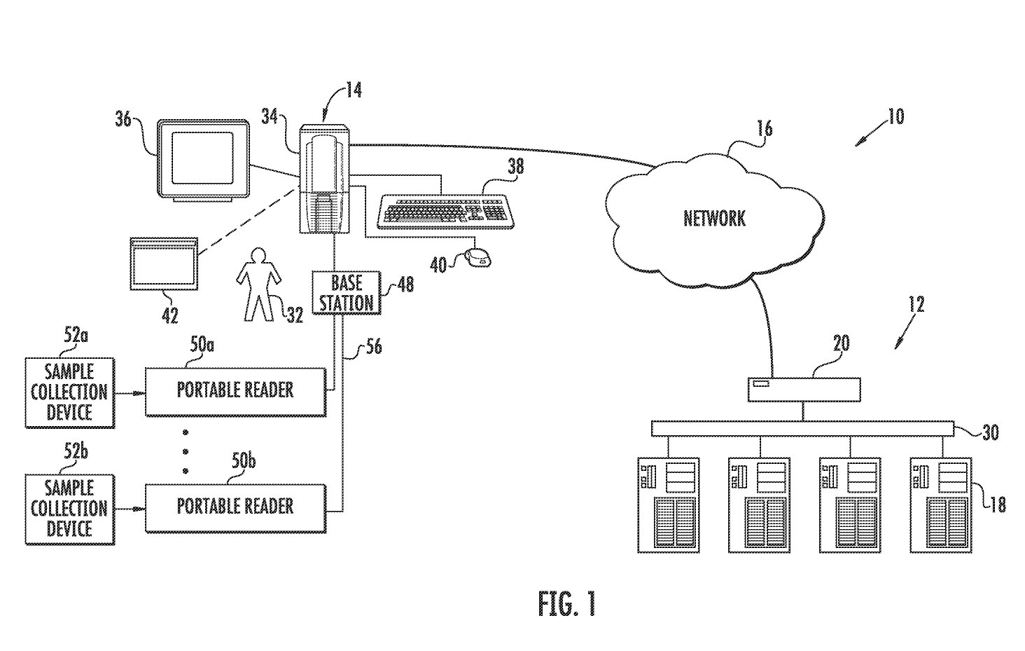

Referring now to the drawings and in particular to FIG. 1, shown therein and designated by reference numeral 10 is a sample analysis system constructed in accordance with the presently disclosed and claimed inventive concept(s). In general, the sample analysis system (referred to hereinafter as the “system 10”) relates generally a system for collecting and analyzing a sample 11 (see FIG. 2) from a patient. The sample 11 can be blood, urine or the like. In particular, the system 10 provides an improved sample analysis system and method that greatly reduces the labor and the likelihood of errors involved in collecting and analyzing the sample 11.

In general, FIG. 1 is an exemplary hardware diagram for the system 10. The system 10 preferably includes a host system 12, communicating with one or more user devices 14 via a network 16. The network 16 can be the Internet, intranet or other network. In either case, the host system 12 typically includes one or more computer systems 18 such as one or more servers, or one or more mainframe computers configured to host or run a medical database and communicate with the network 16 using one or more gateways 20. The medical database can be designed for one hospital/clinic or multiple hospitals/clinics. When the network 16 is the Internet, the primary user interface of the system 10 is delivered through a series of web pages, but the primary user interface can be replaced by another type of interface, such as a Windows-based application permitting users to access or interact with the host system 12 graphically, textually, audio visually, or the like. This method can also be used when the user device 14 of the system 10 is located in a stand-alone or non-portable environment such as a kiosk.

The network 16 can be almost any type of network although the Internet and Internet 2 networks are preferred because of the wide support of their underlying technologies. The preferred embodiment of the network 16 exists in an Internet environment, which means a TCP/IP-based network. However, it is conceivable that in the near future, it may be advantageous for the preferred or other embodiments to utilize more advanced networking topologies. In addition, the network 16 does not refer only to computer-based networks but can also represent telephone communications or other communications.

The computer systems 18 can be networked with a local area network 30. The gateway 20 is one or more entities or devices responsible for providing access between the local area network 30 and the network 16. The gateway 20 can also be used as a security means to protect the local area network 30 from attack from an external network such as the network 16.

The local area network 30 can be based on a TCP/IP network such as an intranet, or can be based on any other suitable underlying network transport technology. The preferred embodiment uses an Ethernet network with TCP/IP because of the availability and acceptance of underlying technologies, but other embodiments may use other types of networks such as Fiber-Channel, SCSI, gigabyte Ethernet, etc.

As discussed above, in one preferred embodiment, the host system 12 includes the computer systems 18. The configuration of the hardware for the computer systems 18 will depend greatly upon requirements and needs of the particular embodiment of the system 10. Typical embodiments, including the preferred embodiment, will include multiple computer systems 18 with load-balancing to increase stability and availability. It is envisioned that the computer systems 18 will include a combination of hardware and software including database servers and applications/web servers. The database servers are preferably separated from the application/web servers to improve availability and also to provide the database servers with improved hardware and storage.

The user device 14 can include any number and type of device. The most typical scenario of the user device 14 involves a user 32, using a personal computer 34 with a monitor 36, a keyboard 38, and a mouse 40. In the preferred embodiment, the user 32 is required to use a type of software called a “browser” as designated by a reference numeral 42. The browser 42 is used to render content that is received from a source, such as the computer systems 18. In the modern vernacular, a “browser” refers to a specific implementation called a Web browser. Web browsers are used to read and render HTML/XHTML content that is generated when requesting resources from a web server. In the preferred embodiment, the system 10 is designed to be compatible with major Web browser vendors such as Microsoft Internet Explorer, Netscape Navigator, Mozilla, Google Chrome, Apple Safari and Opera. However, other embodiments may wish to focus on one particular browser depending upon the common user base connecting to the computer system 18.

The system 10 is designed in this way so as to provide flexibility in its deployment. Depending upon the requirements of the particular embodiment, the system 10 could be designed to work in almost any environment such as a desktop application, a Web based application, or simply as a series of Web services designed to communicate with an external application.

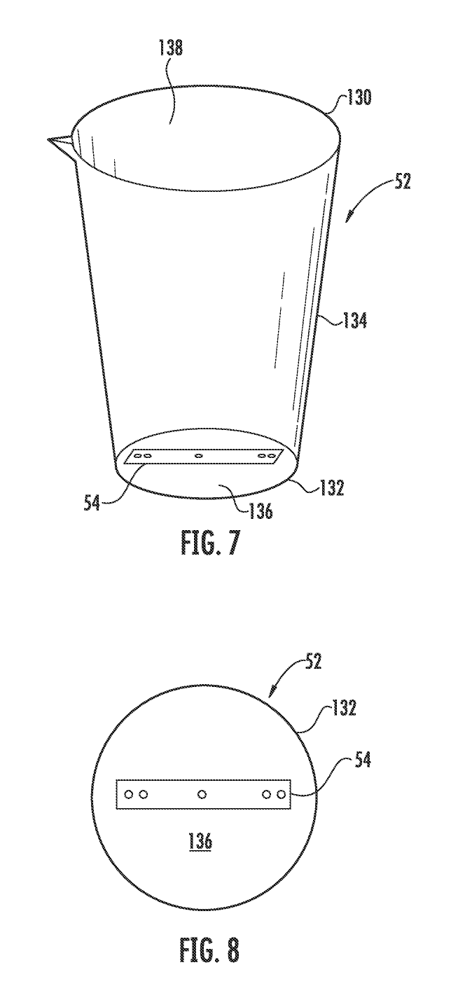

The system 10 also includes one or more base stations 48, one or more portable readers 50a and 50b (generally referred to herein as portable reader(s) 50) for each base station 48 and a plurality of sample collection devices 52a and 52b (generally referred to herein as sample collection device(s) 52). In one embodiment, the base station 48 interfaces with the user device 14 to establish communication there between. For example, the base station 48 can be provided with a USB communication device capable of plugging into a USB port on the user device 14. For purposes of clarity, only two of the portable readers 50a and 50b are shown in FIG. 1, as well as only two of the sample collection devices 52a and 52b. In general, the sample collection devices 52 are disposable after an initial use and include a container 53 to collect and retain the sample 11, and one or more reagent device(s) 54 (shown in FIGS. 7-10) designed to react with the sample 11. Thus, the sample collection devices 52 are designed to collect one or more sample 11 from a patient, preferably directly from the patient, and then to cause one or more reactions between the reagents 54 and the sample 11 that can be detected by one of the portable readers 50 to collect data indicative of the sample 11 as part of a process for analyzing the sample 11.

The portable readers 50 are preferably initialized with a code, such as a patient ID or a lab acquisition ID, indicative of a patient and/or a particular sample 11 prior to collecting data indicative of the specimen, and then communicate the code (or other information related to the code) with the data indicative of the sample 11 and/or patient to correlate the data with a particular sample 11. This is preferably accomplished by establishing communication between the portable readers 50 and the medical database of the host system 12 preferably via networks 56 and 16. The network 56 can be any suitable communication system, such as a wired or wireless system. In a preferred embodiment, the network 56 is a wireless communication system such as those marketed under the names “Bluetooth” or “Wi-Fi”. In one embodiment, the network 56 connects the portable readers 50 with the base station 48 and the user device 14, however, it should be understood that this is optional. In another embodiment, the portable readers 50 could communicate directly with the network 16.

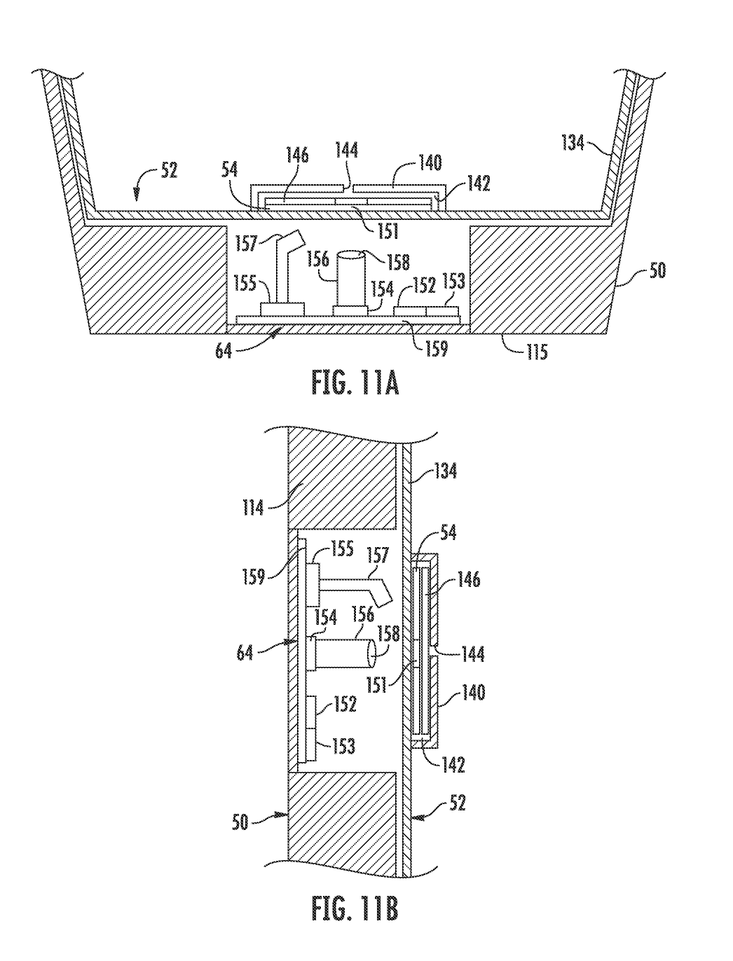

The system 10 generally operates as follows. A user, such as a hospital staff member, utilizes the user device 14 to enter a patient's information from/into the medical database hosted by the computer system(s) 18. The user device 14 also initializes one of the portable readers 50 with the code indicative of the patient or the sample 11 using the base station 48 and the network 56. The user connects one of the sample collection devices 52 to the portable reader 50 to form an assembled device 58 (shown by way of example in FIG. 11) that has been initialized, and then provides the assembled device 58 to the patient. When the sample 11 is urine, the patient goes into a restroom and urinates into the sample collection device 52. As the patient urinates, the portable reader 50 preferably detects the entry of the sample 11 into the sample collection device 52 to trigger a read cycle.

It is also contemplated that the patient would first urinate into the sample collection device 52 and then afterwards place the sample collection device 52 into the portable reader 50 to form the assembled device 58. For example, the portable reader 50 can be fixed to a surface in the restroom and the patients would be instructed to place the cup in the portable reader 50 immediately, i.e., within 5 minutes, after filling the cup. In this embodiment, the portable reader 50 can automatically or manually load information indicative of the patient or the sample from the sample collection device 52 in any suitable manner. For example, the sample collection device 52 can be provided with the unique code in the form of a bar code or an RFID device, which can be read by the portable reader 50.

The sample 11 reacts with the one or more reagents of the one or more reagent device 54, and such reaction(s) are read by the portable reader 50. As can be appreciated, a single sample 11 of liquid can be measured for any desired number of properties at the same time using the one or more reagents of the one or more reagent device 54. For example, a sample of urine could be applied to a chip (discussed below) containing 10 parallel processing channels to test for the presence of nitrate, blood, albumin, specific gravity, creatinine, white blood cells, pH, glucose, ketone, and bacteria at the same time.

There are various reagent methods which could be used in the reagent device 54. Reagents undergo changes whereby the intensity of the signal generated is proportional to the concentration of the analyte measured in the clinical sample 11. These reagents contain indicator dyes, metals, enzymes, polymers, antibodies, electrochemically reactive ingredients and various other chemicals dried onto carriers. Carriers often used are papers, membranes or polymers with various sample uptake and transporting properties. They can be introduced into the reagent wells in the chips of the invention to overcome the problems encountered in analyses using reagent strips. In contrast, reagent strips may use only one reagent area to contain all chemicals needed to generate color response to the analyte. Typical chemical reactions occurring in dry reagent strips can be grouped as dye binding, enzymatic, immunological, nucleotide, oxidation or reductive chemistries.

In some cases, up to five competing and timed chemical reactions are occurring within one reagent layer a method for detecting blood in urine, is an example of multiple chemical reactions occurring in a single reagent device 54. For example, analyte detecting reaction is based on the peroxidase-like activity of hemoglobin that catalyzes the oxidation of a indicator, 3,3′,5,5′-tetramethyl-benzidine, by diisopropylbenzene dihydroperoxide. In the same pad, a second reaction occurs to remove ascorbic acid interference, based on the catalytic activity of a ferric-HETDA complex that catalyzes the oxidation of ascorbic acid by diisopropylbenzene dihydroperoxide.

In particular, the portable reader 50 can conduct readings at set intervals (current timing intervals) for the one or more different reagent devices 54 and then stores the raw data in memory. The portable reader 50 then preferably provides an audible beep (or other indication that the reaction has been read) and the patient dumps the sample 11 (urine) back into the toilet, removes the sample collection device 52 from the portable reader 50, throws away the sample collection device 52 (preferably while still in the restroom), and then hands the portable reader 50 back to the user, e.g., the hospital staff member.

After the read cycle has been conducted, the portable reader 50 automatically uploads the raw data and the code indicative of the patient and/or the sample 11 to the user device 14 via the network 56 and the base station 48. In response thereto, the user device 14 analyzes the raw data to convert it into readable results and uploads the readable results and/or the raw data to the medical database hosted by the host system 12, such as a Laboratory information System or a Hospital Information System or any other electronic medical record system. The user device 14 can also provide other functions, such as preparing a printed report including patient and/or sample information as well as the raw data and/or the readable results.

Thus, the sample analysis system 10 greatly reduces the labor required in collecting and analyzing the sample 11 because the portable reader 50 is initialized prior to the collection of the sample 11, the sample 11 is detected and automatically read, and then the test results are uploaded to the user device 14 and/or the medical database hosted by the host system 12. This reduces or eliminates the need for transferring the sample 11 into one or more separate test tube(s) by the user; the labeling of the test tube(s), storing of the samples for periodic testing and the manual tabulation of the sample results.

Further, the design of the sample analysis system 10 is highly scalable. For example, in a low test volume setting (e.g., a small clinic) a customer would purchase a single base station 48 and a single portable reader 50. As test volume increases, a customer can simply purchase additional portable readers 50. For example, in a medium test volume setting (small hospitals), a customer would purchase a single base station 48 with 2-4 portable readers and in a high volume setting (large hospital and/or clinical laboratory) the customer may need 1-2 base stations 48 with 8-10 portable readers 50.

The design of the sample analysis system 10 allows multiple simultaneous tests to be conducted and is limited only by the number of base stations 48, portable readers 50 and available sample collection facilities, such as restrooms, bedsides, doctor's offices or the like. Further, the test results can be logged into the system 10 almost in real-time; the patient's test results are preferably analyzed and uploaded to the medical database as soon as a patient returns the portable reader 50. For the large hospital, this design can dramatically reduce the workload needed for urinalysis tests. In one embodiment, the system 10 completely eliminates the time consuming sample collection, transfer (from cups into test tubes), accumulation and bar coding steps. In a clinical laboratory, the system 10 can eliminate the step of transferring the sample from the cups into test tubes by having the lab personnel place the sample collection device 52 into or on the portable reader 50 to read the sample within the sample collection device 52.

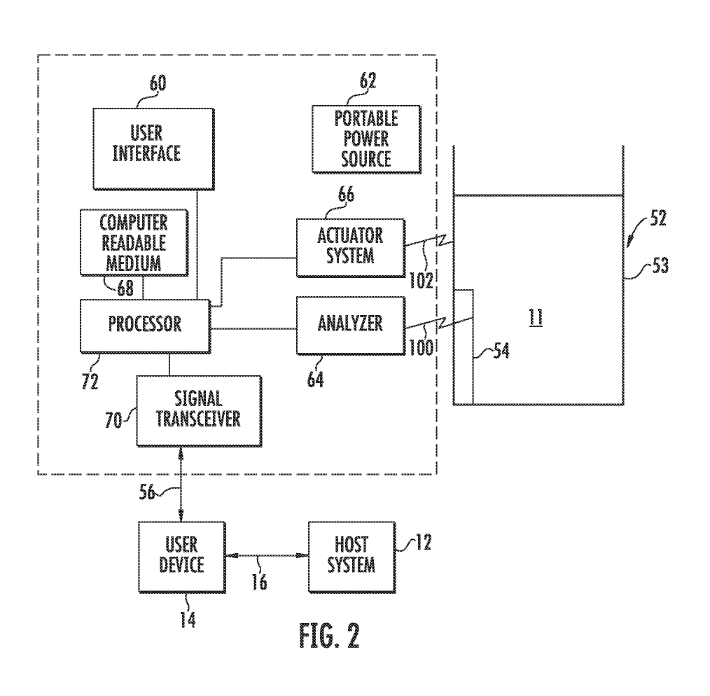

Referring now to the drawings, and in particular to FIG. 2, shown therein is a schematic view of the sample analysis system 10 of FIG. 1 showing a block diagram of an exemplary portable reader 50. In general, the portable reader 50 is provided with one or more user interface 60, one or more portable power source 62, one or more analyzer 64, one or more actuator system 66, one or more computer readable medium 68, one or more signal transceiver 70, and one or more processor 72. FIG. 3 is a logic flow diagram of logic stored on the computer readable medium 68, that when executed by the one or more processor 72 causes the one or more processor 72 to execute the steps of the process.

In particular, the processor 72 is programmed with logic, preferably stored as computer executable instructions on the one or more computer readable medium 68, which permits the portable reader 52 to be: initialized with the code identifying the patient and/or the sample 11 (as indicated by a block 80); communicate with the patient and/or the user via the user interface 60 to provide an indication that the portable reader 50 has been initialized (as indicated by a block 82); detect the presence of the sample 11 with input from the actuator system 66 (as indicated by a block 84); enable a read cycle to detect chemical reactions between the sample 11 and the reagent device 54 via the one or more analyzer 64 (as indicated by a block 86); store the raw data detected by the one or more analyzer 64 on the one or more computer readable medium 68 (as indicated by a block 88); and upload the raw data and the code to the user device 14 and/or the host system 12 utilizing the signal transceiver 70 (as indicated by a block 90) as discussed above.

The user interface 60 can be any suitable type of device or devices capable of communicating with the patient and/or the user. For example, the user interface 60 can include one or more speakers, beepers, light sources, such as an LED or an LCD display, or the like for notifying the patient and/or the user of the current or expected status of the portable reader 50.

The portable power source 62 can be one or more devices capable of supplying power to electronic devices of the portable reader 50, such as the processor 72, the user interface 60, the analyzer 64, the actuator system 66, the computer readable medium 68, the signal transceiver 70, and the processor 72. The portable power source 62 can be implemented in a variety of ways including a power storage device, such as a Li-ion battery, and/or a device capable of converting movement into electrical power.

The analyzer 64 is adapted to communicate with the reagent device 54 as indicated by the reference numeral 100 so as to detect the results of a reaction which has occurred between one or more portions of the reagent device 54, and the sample 11. The analyzer 64 can be implemented in a variety of manners such as an optical reader, and/or an electrochemical reader. The analyzer 64 can include one or more sensors that are either fixed or movable for providing interrogating radiation to the sample-reagent combination and also for receiving signals indicative of turbidimetric, fluorometric, absorption readings or the like. The analyzer 64 can also include a motor, an actuator and/or a track system for sweeping the one or more sensors through a predetermined field of view to read various parts of the reagent device 54. One of ordinary skill in the art would clearly appreciate how to make and use conventional optical readers and electrochemical readers. Thus, a detailed discussion of how to make and use the optical reader and the electrochemical reader is not necessary to teach one skilled in the art how to make and use the portable reader 50.

The analyzer 64 used to analyze the reacted sample may be any system, subsystem, and/or component suitable for detecting light or any other signal from the sample. The analyzer 64 may detect and/or sense the magnitude of light or other wavelengths of electromagnetic radiation. For example, the analyzer 64 may return a result corresponding to the intensity of the light sensed by the analyzer 64. In exemplary embodiments, the analyzer may include, but is not limited to, a photo diode, a charge coupled device (CCD) imager, or an electrochemical analyzer such as a CMOS analyzer. The analyzer 64 may return a result corresponding to a color value associated with the light. For example, the analyzer 64 may return a result corresponding with the wavelength of light sensed by the analyzer 64. In one embodiment, the analyzer 64 may detect a luminance value associated with the magnitude of the intensity of the light sensed by the analyzer 64.

The actuator system 66 is designed to interface with the sample collection device 52 as shown by the reference numeral 102 for generating signals indicative of the entry of the sample 11 into the sample collection device 52. This can be accomplished in a number of ways depending upon the configuration of the sample collection device 52, and/or the sample 11. When the sample collection device 52 resembles a cup, as shown in FIG. 2, and when the sample 11 being collected is urine, the actuator system 66 can be implemented either as a thermocouple for detecting a change in temperature based upon entry of the sample 11 into the sample collection device 52, and/or the actuator system 66 can include a spring for detecting a difference in the mass of the sample collection device 52. The actuator system 66 can be implemented in other ways, such as with one or more devices working together to detect an electrochemical change (e.g., impedance, or capacitance) or an optical change (e.g., reflectance, luminescence or absorbance) and the thermocouple and the spring are discussed herein by way of example. The data generated by the actuator system 66 indicative of the presence of the sample 11 from either a change in temperature or a change in mass, for example, is provided to the processor 72. The processor 72 is programmed to monitor the data from the actuator system 66 and to automatically enable the read cycle, (preferably without any patient intervention) either immediately or within a predetermined time period, upon detection of the presence of the sample 11. The detection of the presence of the sample 11 can be determined in various manners, such as by looking for a rapid transition in the data, or by detecting a change in the data exceeding a predetermined rate.

The computer readable medium 68 can be implemented in a variety of ways, such as a memory (either on board in the processor 72, or external thereto), a hard disk (mechanical, magnetic, and/or solid-state), a removable disk, or the like. In general, it is envisioned that the entire circuitry of the portable reader 50 will be contained within a housing 104 of the portable reader 50. However, it should be understood that this does not have to be the case—especially with respect to the computer readable medium 68. The computer readable medium 68 can either be fixed within the housing 104 of the portable reader 50, or can be removable therefrom. For example, the computer readable medium 68 can be implemented as a portable device known in the art as a “jump drive”.

The signal transceiver 70 is adapted to communicate bi-directionally either to and/or from the user device 14 via the network 56, and/or to the host system 12 via the network 56, the base station 48, the user device 14, and the network 16. Alternatively the signal transceiver 70 can communicate with the network 16 using the base station 48 thereby bypassing the user device 14. The signal transceiver 70 can be implemented in a variety of manners and in a preferred embodiment is a bidirectional wireless transceiver. It should be noted that the signal transceiver 70 is an optional element. For example, when the computer readable medium 68 is implemented as a removable device, the initialization of the portable reader 50 and the collection of raw data therefrom can be implemented using the computer readable medium 68 rather than the signal transceiver 70. The initialization of the portable reader 50 can be accomplished by loading the code onto the computer readable medium 68 by the user device 14, for example, and then plugging the computer readable medium 68 into the portable reader 50. Likewise, the downloading of the raw data can be accomplished by storing the raw data onto the computer readable medium 68, removing it from the portable reader 50 and then plugging it into the user device 14.

The processor 72 of the portable reader 50 can be implemented in a variety of manners, such as one or more central processing unit, microcontroller, digital signal processor, or the like. In general, the processor 72 can be implemented as one or more devices adapted to read computer executable instructions to cause the processor 72 to implement the functions provided by the computer executable instructions. Of course, the processor 72 will be provided with a variety of input and output ports for interfacing with the user interface 60, the analyzer 64, the actuator system 66, the computer readable medium 68, and the signal transceiver 70.

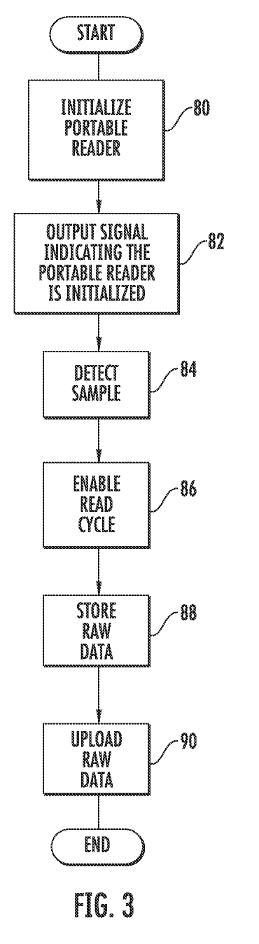

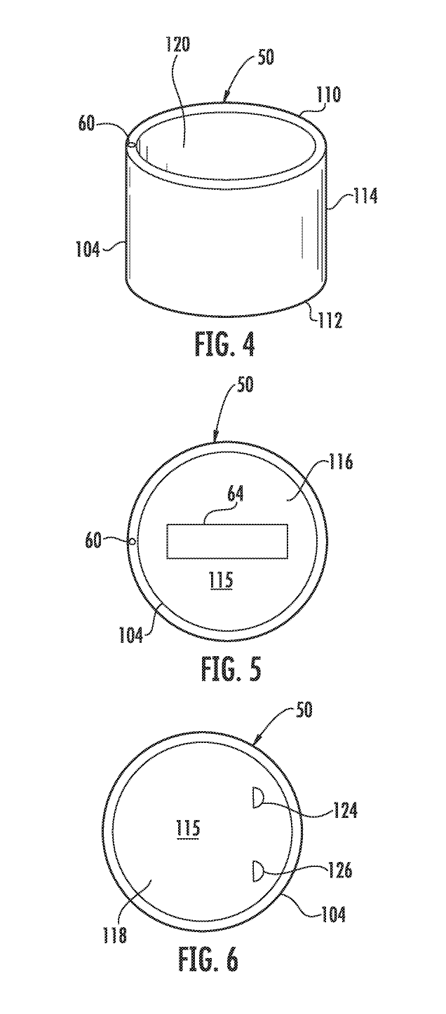

Referring now to FIGS. 4-6, shown therein is an exemplary embodiment of the portable reader 50 constructed in accordance with the presently disclosed and claimed inventive concept(s). In this embodiment, the portable reader 50 is provided with the housing 104 having an upper end 110, a lower end 112, a side wall 114 extending between the upper end 110 and the lower end 112, and a bottom 115 positioned generally at the lower end 112. The bottom 115 has an inner surface 116, and an outer surface 118. The side wall 114 and the inner surface 116 of the bottom 115 cooperate to define a space 120 which is sized and adapted to receive at least a portion of the sample collection device 52. As shown in FIGS. 4, 5 and 6, the user interface 60 is preferably provided on the side wall 114, near the upper end 110 thereof. However, other locations can be used.

It should be understood that the portable reader 50 can be constructed in a variety of manners and the above description is merely by way of example. In the portable reader 50 shown in FIGS. 4-6, the sidewall 114 is used to register the portable reader 50 with the sample collection device 52, however, it should be understood that the sidewall 114 is optional, and other manners of registering the portable reader 50 with the sample collection device 52 can be used, such as nubs or posts extending from the bottom 115 to engage predetermined recesses formed in the sample collection device 52.

As shown in FIG. 5, the analyzer 64 can be positioned in the bottom 115 of the portable reader 50. The sample collection device 52 can be provided with a cover (not shown) to provide protection to the analyzer 64. As shown in FIG. 6, the power source 62 can be provided with battery charging contacts 124 and 126 for establishing contact with the battery charging contacts (not shown) provided on the base station 48.

In the embodiment depicted in FIGS. 5 and 11A, the bottom 115 supports the analyzer 64 such that it is located at a position beneath the sample collection device 52 to analyze the reagent device 54. However, in other versions, the portable reader 50 can be designed as a sleeve and so the bottom 115 is an optional feature of the sample collection device 52. In these versions, the analyzer 64 can be supported by the sidewall 114 as depicted in FIG. 11B.

Referring now to FIGS. 7, 8 and 9, shown therein is an exemplary embodiment of the sample collection device 52. In particular, the container 53 of the sample collection device 52 is provided with an upper end 130, a lower end 132, a sidewall 134 extending generally between the upper end 130 and the lower end 132, and a bottom 136 positioned either at or near the lower end 132. In general, the sidewall 134 and the bottom 136 form the container 53 and function to define a collection space 137 (FIG. 9) for receiving and retaining at least a portion of the sample 11. The volume of the collection space 137 can vary between about 10 mL to 3000 mL, but typically such collection space 137 will have a volume between about 75 mL to about 200 mL, and more typically about 100 mL. The volume of the collection space 137 can depend upon a variety of factors, such as whether the sample 11 will be collected at a single time, or multiple times. Further, the sidewall 134 defines an opening 138 generally near the upper end 130 for receiving the sample 11 into the collection space 137.

As best shown in FIG. 8, the reagent device 54 is positioned on the container 53 in any suitable location such that the reagent device 54 can contact and react with the sample 11 and be read by the portable reader 50. For example, the reagent device 54 can be positioned on the bottom 136 of the sample collection device 52; however, it should be understood that the reagent device 54 can also be positioned on the sidewall 134. The sample collection device 52 is also provided with a retaining member 140 which is connected to the bottom 136 (for example) and extends over and encapsulates the reagent device 54 to form a reaction chamber 142 surrounding the reagent device 54. The retaining member 140 can be connected to the bottom 136 in any suitable manner, such as by RF welding. The retaining member 140 also defines at least one opening 144 that provides access to the reaction chamber 142 so that at least a portion of the sample 11 can contact and thereby interact with the reagent device 54. The retaining member 140 can be provided with one opening 144 such that the sample 11 enters the reaction chamber 142 by capillary action.

The volume of the reaction chamber 142 can vary widely between about 10 μL to about 1200 μL, and is usually in a range from about 10 μL to about 40 μL. The volume of the reagent device 54 can also vary widely between about 5 μL to about 600 μL, and is usually in a range from about 5 μL to about 20 μL. The sample volume can vary, but typically, such samples have volumes of about 3 μL to 20 μL per reagent, although they may range from 0.1 μL to 200 μL per reagent depending on the type of sample and the number of metering steps. When the sample is urine, the sample volume will typically be about 10 μL.

A ratio of the volume of the collection space 137 to the reaction chamber 142 can vary widely and be between about 8.33:1 to about 300,000:1; and more preferably between about 2,500:1 to about 10,000:1 and even more preferably about 5,000:1 to about 7,500:1.

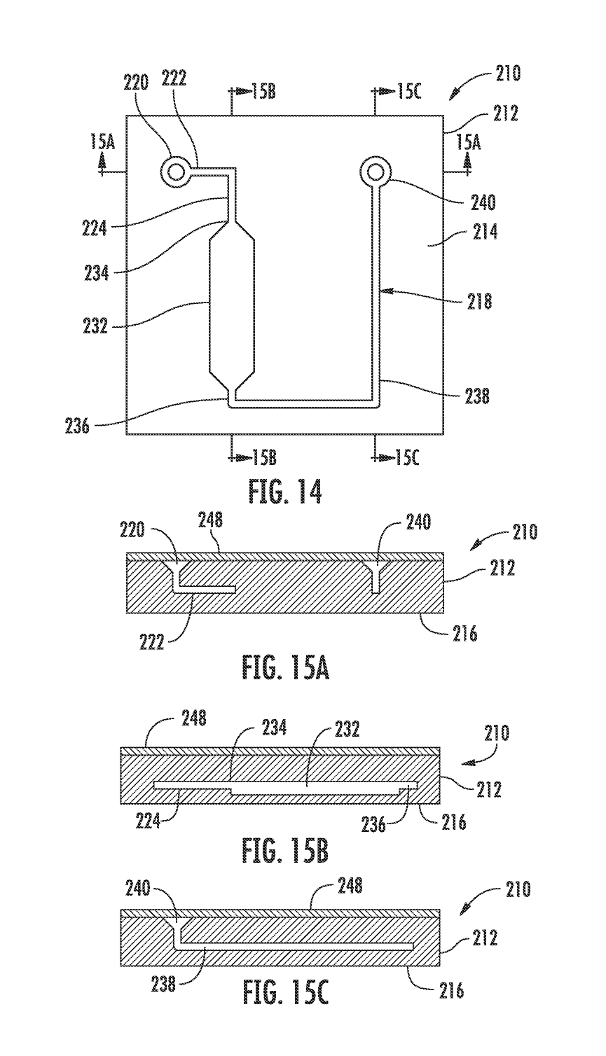

The retaining member 140 having one opening 144 is optional and in an alternative embodiment, the retaining member 140 can define at least two openings 144 with at least one of the openings 144 forming a vent to facilitate the sample 11 entering into the reaction chamber 142. Embodiments having more than one openings 144 are described hereinafter with reference to FIGS. 14-32.

The bottom 136 of the container 53 is preferably constructed of a material which is transparent to the type of radiation which is being emitted by the analyzer 64 and also transparent to any fluorescence, reflection, or other information which is generated by the reagent device 54 in response to receiving the radiation from the analyzer 64 so that the information indicative of the reaction can pass through the bottom 136 and be received by the analyzer 64. The bottom 136 can be made of plastics such as polycarbonate, polystyrene, polyacrylates, or polyurethane, alternatively, they can be made from silicates, and/or glass. When moisture absorption by the plastic is not a substantial concern, the plastics preferably used may include, but are not limited to, ABS, acetals, acrylics, acrylonitrile, cellulose acetate, ethyl cellulose, alkylvinylalcohols, polyaryletherketones, polyetheretherketones, polyetherketones, melamine formaldehyde, phenolic formaldehyde, polyamides (e.g., nylon 6, nylon 66, nylon 12), polyamide-imide, polydicyclopentadiene, polyether-imides, polyethersulfones, polyimides, polyphenyleneoxides, polyphthalamide, methylmethacrylate, polyurethanes, polysulfones, polyethersulfones and vinyl formal. When moisture absorption is of concern, preferably the plastics used to make the chip include, but are not limited to: polystyrene, polypropylene, polybutadiene, polybutylene, epoxies, Teflon™, PET, PTFE and chloro-fluoroethylenes, polyvinylidene fluoride, PE-TFE, PE-CTFE, liquid crystal polymers, Mylar®, polyester, LDPE, HDPE, polymethylpentene, polyphenylene sulfide, polyolefins, PVC, and chlorinated PVC.

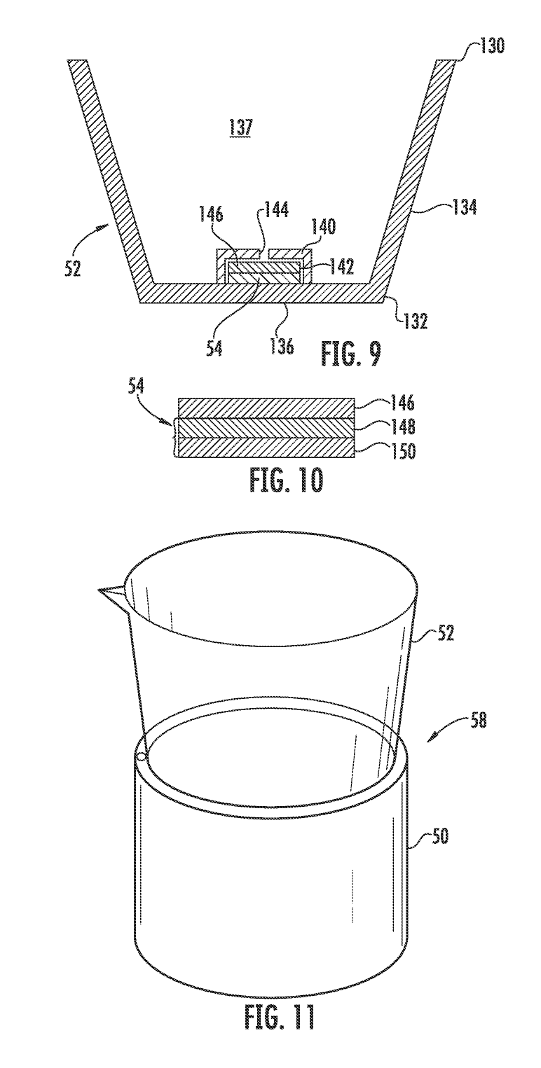

When the sample collection device 52 is intended to be used in conjunction with an optical reader, the sample collection device 52 is also provided with a shield 146 positioned adjacent to the reagent device 54 to shield the analyzer's optics from background lights or other radiation during testing. The shield 146 can be provided in a variety of manners, such as by providing a backing on the reagent device 54 as shown in FIGS. 9 and 10. The backing can be black polyester, for example.

One example of the reagent device 54 is depicted in FIG. 10. In this example, the reagent device 54 is constructed as a three-layer structure having a reagent substrate 148, positioned in between the shield 146 and a double-sided adhesive layer 150. The shield 146 and the double-sided adhesive layer 150 can be pre-punched with a suitable shape, such as circles, to expose the reagent substrate 148 so that the sample 11 will wick into the reagent substrate 148 and the air pressure within the reaction chamber 142 will prevent an excess of sample 11 build-up within the reaction chamber 142. The double-sided adhesive layer 150 serves to connect the reagent device 54 to the bottom 136 of the container 53 while permitting the reagent device 54 to be read from a position beneath the container 53, e.g., through the bottom 136 of the container 53. When the analyzer 64 of the portable reader 50 is an optical reader, then the double-sided adhesive layer 150 can either be optically transparent, or optically opaque with cutouts aligned with predetermined portions of the reagent device 54 to permit optical inspection of the reagent device 54.

It should also be understood that the positions of the reagent device 54 within the container 53 and the analyzer 64 within the portable reader 50 are predetermined and matched so that the reagent device 54 is positioned adjacent to the analyzer 64 when the sample collection device 52 is installed on the portable reader 50. It should also be understood that the sample collection device 52 can be provided with multiple reagent devices 54 and a retaining member 140 for each reagent device 54; and the portable reader 50 can be provided with multiple analyzers 64 with one or more of the analyzers 64 for each reagent device 54. It should also be understood that the reagent device 54 can be provided separately from the container 53 and collect sample 11 therefrom using any suitable system of connecting device(s), port(s) and/or vent(s).

Shown in FIG. 10 is one embodiment of the assembled device 58. In this embodiment, the assembled device 58 is formed by positioning the lower end 132 of the container 53 into the collection space 137 of the portable reader 50 to align the reagent device 54 with the analyzer 64. Preferably, the portable reader 50 and the container 53 of the sample collection device 52 are adapted to be connected together so that the assembled device 58 does not inadvertently come apart, to retain the alignment of the reagent device 54 with the analyzer 64, and to also form a sealed environment for the analyzer 64. This can be accomplished in a variety of ways, such as using snaps, magnets, screw threading, friction retainers, keys, interlocking grooves or the like.

FIG. 11a depicts an exemplary analyzer 64 for measuring color response of a test area 151 of reacted reagent on the reagent device 54. The analyzer 64 is positioned in the bottom 115 of the portable reader 50. The analyzer 64 may include a processor 152 in connection with a datastore 153, a detector 154, and a light source 155. The analyzer 64 may include a receiver optical unit 156 coupled with the detector 154. The analyzer 64 may also include an illumination optical unit 157 coupled with the light source 155.

Light from the light source 155 and directed by the illumination optical unit 157 may reflect off of the surface of the test area 151. The light reflected from the test area 151 may correspond with the color response of the test area 151. The light reflected from the test area 151 may be within a field of view, as defined by the receiver optical unit 156 and/or the detector 154. The light reflected from the test area 151 may reach and/or be sensed by the detector 154. The detector 154 may measure the color and/or intensity of the light received.

The processor 152 may be any system, subsystem, and or component suitable for processing data and/or controlling the detector 154 and/or the light source 155. The processor 152 may be a microprocessor, a microcontroller, a collection of logical hardware components, and the like. The processor 152 may direct the light source 155 to illuminate. The processor 152 may direct the detector 154 to sense light. The processor 152 may receive a reading from the detector 154 corresponding to the light sensed by the detector 154. The processor 152 may be connected to the datastore 153. The processor 152 may store readings received from the detector 154 at the datastore 153. The processor 152 may receive computer executable instructions from the datastore 153. The computer executable instructions may direct the processor 152 to operate and/or control the detector 154 and/or the light source 155.

The light source 155 may be any system, subsystem, and or component suitable for generating light. For example. the light source 155 may be a light emitting diode (LED). Also for example, the light source 155 may be an incandescent light, fluorescent light, halogen light, and the like. The light source 155 may be an array of LEDS. The light source 155 may be controlled by the processor 152. The light source 155 may receive instructions from the processor 152 to illuminate according to a timing defined by the processor 152.

The light source 155 may be coupled with the illumination optical unit 157. The illumination optical unit 157 may be any system, subsystem, and/or device suitable for directing in light from the light source 155 to the test area 151. The illumination optical unit 157 may provide a substantially uniform distribution of light from the light source 155 across the test area 151. For example, the illumination optical unit 157 may be a light guide, a lightbox, an optical fiber, a conventional lens, a total internal reflection lens, and the like. For example, the illumination optical unit 157 may be a light guide with a circular cross-section, and/or a rectangular cross-section.

The detector 154 may be any system, subsystem, and/or component suitable for detecting light. The detector 154 may detect and/or sense the magnitude of light. For example, the detector 154 may return a result corresponding to the intensity of the light sensed by the detector 154. In an embodiment, the detector 154 may be a photo diode. In an embodiment, the detector 154 may be a charge coupled device (CCD) imager which takes a picture of the test area 151. The detector 154 may return a result corresponding to a color value associated with the light. For example, the detector 154 may return a result corresponding with the wavelength of light sensed by the detector 154. In an embodiment, the detector 154 may detect a luminance value associated with the magnitude of the intensity of the light sensed by the detector 154. In an embodiment the analyzer 64 may determine a reading for a plurality of wavelengths by directing the light source 155 to illuminate the plurality of wavelengths. The detector 154 may sense a luminance value associated with the respective wavelength. The processor 152 may coordinate the sequence of wavelengths illuminated by the light source 155 and/or the corresponding sequence of readings received from the detector 154.

The detector 154 may be coupled with the receiver optical unit 156. The receiver optical unit 156 in combination with the detector 154 may define a field of view. The field of view may define the scope of light that reaches the surface of the detector 154 and/or be sensed by the detector 154. The receiver optical unit 156 may include an aperture 158 which may or may not be opened and/or closed by a shutter (not shown). The aperture 158 may limit the amount of light that may reach the detector 154. The receiver optical unit 156 may be a light guide, an optical fiber, an axicon, an imaging lens, and the like.

In an embodiment, the light source 155 may include an array of light emitting diodes having an area of about 0.44 mm by 0.51 mm (+/−0.2 mm) and/or the area equivalent. The illumination optical unit 157 may include a light guide having a cross-sectional area of about 2.7 mm by 2.7 mm (+/−0.5 mm) and/or the area equivalent.

The light source 155, detector 154, processor 152, and datastore 153 may be connected to one or more elements 159 such as a circuit board(s) and/or cables to permit electrical communication therebetween while also providing mechanical support to maintain the light source 155, the detector 154, the processor 152 and the datastore 153 securely within the bottom 115 of the portable reader 50.

Shown in FIG. 11b is another embodiment of the portable reader 50 having the analyzer 64 (described above in connection with FIG. 11a incorporated into the sidewall 114 thereof. In this embodiment, the reagent device 54 extends along the sidewall 134 of the sample collection device 52 so as to be aligned (or colinear) with the test area 151 of the reagent device 54.

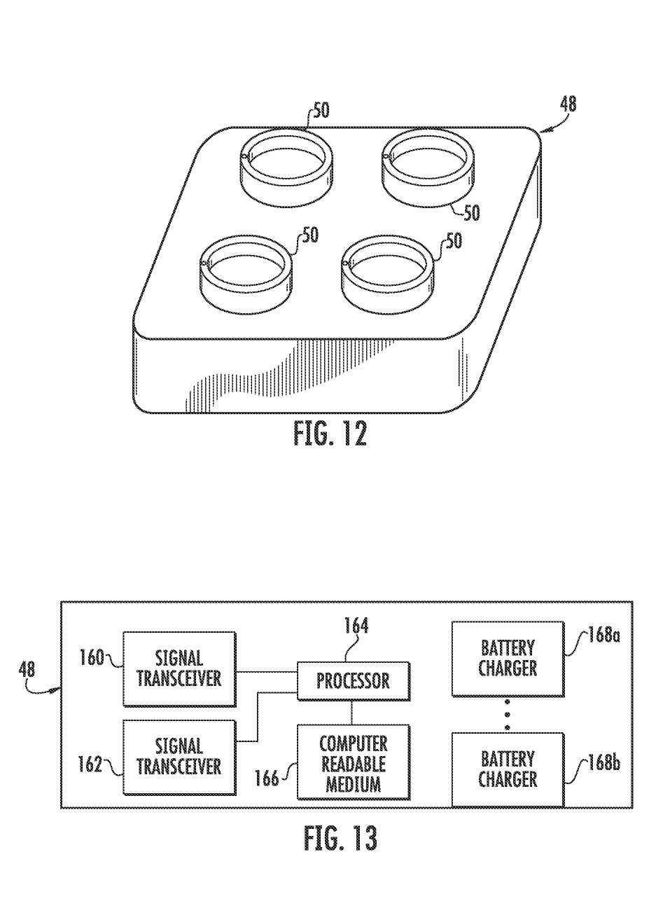

Shown in FIGS. 12 and 13 is an exemplary base station 48 constructed in accordance with the present invention. In general, the base station 48 serves as a communication hub to establish communication between one or more portable readers 50 and the user device 14; and as a charging platform for the portable readers 50 when not in use. The base station 48 and the portable readers 50 can be adapted with suitable communication schemes to ensure that only predetermined portable readers 50 can be recognized and communicate with the base station 48.

In this embodiment, the base station 48 is provided with two signal transceivers 160 and 162; a processor 164; one or more computer readable medium 166; and one or more battery charger(s) 168a and 168b (which are generally referred to using the reference numeral 168). In the example shown, the base station 48 is provided with four battery chargers 168, with each of the battery chargers 168 connected to and charging a battery of one of the portable readers 50. Alternatively, the base station 48 can be provided with one battery charger 168 having multiple charging ports for charging the batteries of multiple portable readers 50. The two signal transceivers 160 and 162 are preferably of different types, however, the signal transceivers 160 and 162 can be of a same type. For example, the signal transceiver 160 can be wired connection for connecting to the user device 14, such as a USB communication device; and the signal transceiver 162 can be a wireless communication device, such as those commonly sold under the names “Bluetooth” and “Wi-Fi”; both of which are well known to those skilled in the art. Alternatively, the base station 48 can be provided with one of the signal transceivers 160 or 162 for communicating with the user device 14 and the portable readers 50.

Preferably, computer executable instructions for enabling operation of the base station 48 are stored in the computer readable medium 166 and then uploaded to the user device 14 using the signal transceiver 160. The computer executable instructions can include data analysis algorithms for converting the raw data collected by the portable readers 50 into readable results, as discussed above. This can be automatically accomplished when the signal transceiver 160 is connected to the user device 14, or can be manually accomplished thereafter. Preferably, the base station 48 is adapted to provide the computer executable instructions to the user device 14 (for execution by a processor of the user device 14) to cause the user device 14 to (1) convert the raw data into the readable results, and (2) upload the readable results to the medical database of the host system 12. It should be understood that the host system 12 can be programmed with computer executable instructions to cause the host system 12 to (1) convert the raw data into the readable results, and (2) enter the readable results into the medical database. In this instance, the computer executable instructions will be stored on a computer readable medium (not shown) accessible by the host system 12 and executed by one or more processors (not shown) of the host system 12.

To minimize cost, the portable reader 50 has been described as storing the raw data that it receives from the analyzer 64 and then transmitting the raw data to the user device 14 and/or the host system 12 to convert the raw data into readable results which can then be stored in the medical database, and/or provided on a written report as discussed above. However, the portable reader 50 can be provided as a more robust system for converting the raw data collected by the analyzer 64 into readable results, storing the readable results and then transmitting or uploading the readable results to the host system 12, user device 14, and/or the base station 48. This can be accomplished by storing computer executable instructions on the computer readable medium 68 indicative of data analysis algorithms, that when executed by the processor 72 converts the raw data into the readable results.

Throughout this document, the words user, or customer are generally used interchangeably to indicate a person associated with a data collection or analysis facility, such as a clinic, lab or hospital unless otherwise indicated.

It should be understood that the various components of the presently disclosed and claimed invention can be provided as kits containing various combinations of the components that can be assembled or used by the user and/or patient in the manners disclosed above. For example, the assembled device 58 can be provided as a kit including one or more sample collection device 52 and one or more portable reader 50 that can be assembled and used by the user and/or patient.

Examples of Sample Collection Devices

As discussed previously, the sample collection device 52, which can be used to support the reagent device 54 in a predetermined position to be read by the portable reader 50, may be constructed in a variety of manners. Discussed below are various examples of sample collection devices which are constructed using one or more microfluidic system and which are suitable for use in a similar manner as the sample collection device 52 discussed above.

As noted previously, POC testing systems are becoming continuously smaller which leads to problems with features such as constructing microfluidic systems, detecting and reading reaction results therein, and delivering adequate sample size. In accordance with the presently claimed and disclosed inventive concept(s) in order to have a microfluidic system which functions optimally, the following elements are preferably combined in a single system: the fluidics should be connected by a lay user without error; the sample collection device should not generate air gaps which interrupt operation; the portable reader 50 should not be contaminated between sample collections; samples and reagent waste are bio-hazards and should be disposed of; and the sample collection device and portable reader 50 is preferably able to work in at least several, if not all, orientations.

An important part of the solution to the problems addressed herein, and as described below, can be the integration of microfluidic devices directly into sample collection. This integration allows biohazard and reagent waste to be contained in a disposable item, i.e., the sample collection device, for easy removal and prevents contamination of a larger system with sample. Environmental waste is reduced by not requiring separate collection and reagent devices.

Piezoelectric reagent dispensers and CMOS electrochemical analyzers may also be integrated with the microfluidic device as reuse-able cartridges that can be easily connected and disconnected. The micro-optics (MORH) may be integrated in the analyzer 64 of the reader 50 for reading the reagent device 54 of the sample collection device 52. This allows all benefits of these technologies to be realized while decreasing system size and per assay cost.

The following is a general description of techniques for implementing sample collection devices as described in more detail below. In a preferred embodiment of the presently claimed and disclosed inventive concept(s), a container of the sample collection device and a microfluidic device containing reagents are separate and connectable to form the sample collection device. The user or a technician may connect the container of the sample collection device to the microfluidic device having one or more reagents for analyzing a sample.

The sample collection device may include a container such as a cup, a capillary tube, or any other sample collection device. For example, sample collection devices include a transfer capillary filled with blood or urine and/or a urine cup with an air vent capillary. The transfer capillary, for example, connects to a sample inlet port on the microfluidic device. The sample may be transferred into the microfluidic device by a pushing force such as with a plunger, by capillary force caused by opening an air vent on the urine cup, or by drawing by a pulling force.

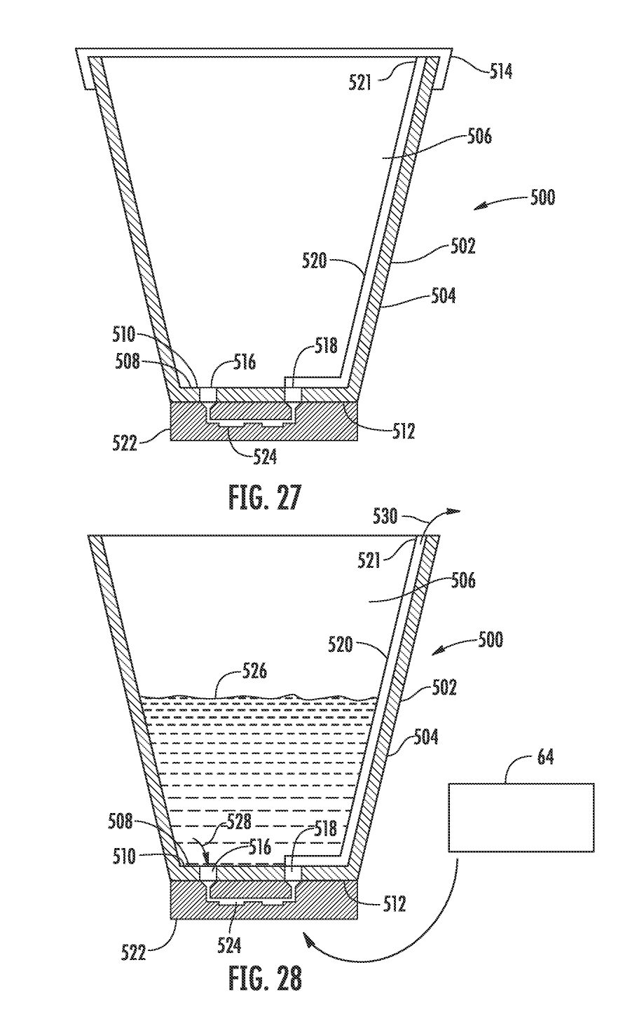

In one embodiment, the principle of operation of the system of the presently claimed and disclosed inventive concept(s) is that the sample is provided to a reagent in a reaction chamber through the use of a unidirectional hydrophilic capillary flow principle where the sample flows from a sample entry port, through the reaction chamber, towards an exit air vent (an example of which is shown in FIGS. 27 and 28 and referred to as an “air capillary 520”). The vent is open to air during flow. Flow does not occur while the vent is not open to air. This principle can be used for timing reactions by starting flow at a known time when the vent is opened. Sealing the air vent prevents flow into the reaction chamber and opening the vent starts flow. A simple means of opening a vent may be through puncturing or removing a sealing device over the vent or simply removing a lid 514 of the device. For example, flow could be started by removing the lid 514 when the sample collection device is connected to the portable reader 50, or the vent could be opened after the lid 514 is removed, i.e., the vent would be sealed with a sealing device, such as tape, that is separate from the lid 514.

The inlet port, in one embodiment, is connected to a sample chamber by a capillary passageway, also referred to herein as a microconduit. Air is purged from an air vent upstream from an inlet port into the sample chamber. An overflow chamber may be used to assure complete filling. Once filled, the input port may be blocked by flow from the overflow chamber and flow towards the air vent.

Described herein, and shown in the accompanying figures, are several non-limiting embodiments of sample analysis systems and microfluidic devices of the presently claimed and disclosed inventive concept(s) which may be used for analyzing a liquid sample according to the presently claimed and disclosed inventive concept(s). Preferably the liquid sample is from a biological source. A “liquid” refers to any substance in a fluid state having no fixed shape but a substantially fixed volume.

The microfluidic devices of the sample collection device of the presently claimed and disclosed inventive concept(s) typically use smaller channels (referred to herein as microconduits) than have been proposed by previous workers in the field. In particular, the channels (microconduits) used in the presently claimed and disclosed inventive concept(s) typically have widths in the range of about 10 to 500 μm, preferably about 20-100 μm, whereas channels an order of magnitude larger have typically been used by others when capillary forces are used to move fluids. Depths of the microconduits are typically in a range of 5 μm to 100 μm. The minimum dimension for the microconduits is preferably to be about 5 μm, since smaller channels may effectively filter out components in the sample being analyzed. Channels in the range preferred in the presently claimed and disclosed inventive concept(s) make it possible to move liquid samples by capillary forces alone. It is also possible to stop movement by capillary walls that have been treated to become less hydrophilic (or hydrophobic) relative to the sample fluid. As noted herein, the resistance to movement can be overcome by a pressure difference, for example, by applying centrifugal force, pumping, vacuum, electroosmosis, heating, or additional capillary force. As a result, liquids can move from one region of the device to another as required for the analysis being carried out.

The microfluidic devices of the sample collection device of the presently claimed and disclosed inventive concept(s), also referred to herein as “chips” or “microfluidic chips”, are generally small and flat, typically about 1 to 2 inches square (25 to 50 mm square) or disks having a radius of about 20 to 80 mm. The volume of samples introduced into the microfluidic circuit will be small. For example, they will contain only about 0.1 to 10 μL for each assay, although the total volume of a specimen may range from 10 to 200 μL. The reaction chambers for the sample fluids (and sample chamber and overflow chambers where present) will be relatively wide as compared to the microconduits in order that the samples can be easily seen and changes resulting from reaction of the samples can be measured by suitable equipment as described herein.

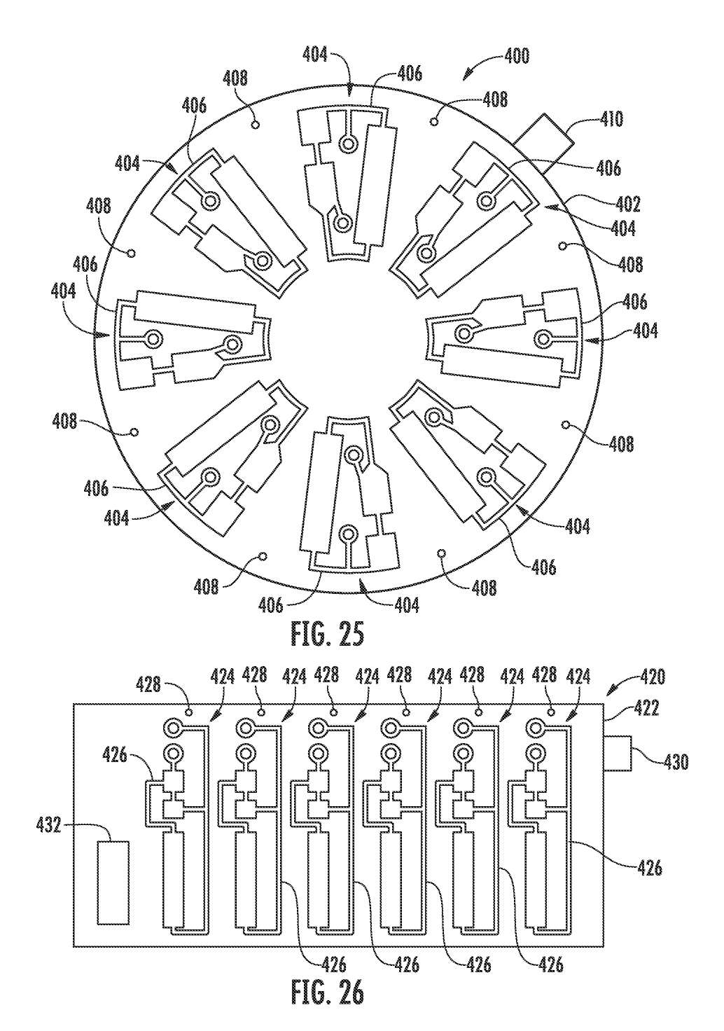

The base or substrate material used to make the microfluidic devices, generally made of a plastic material, is preferably about 1 to 8 mm thick to keep moisture transfer below 0.01 mg of water added for each 1 mg of dry reagent over the shelf life of the device. However, the devices are typically made by cutting or molding the desired features into the base (substrate) and then covering the surface through which the features were cut or molded with a cover portion comprising a relatively thin layer of a film or plastic to complete the device. This cover portion may be attached with an adhesive, or other bonding mechanisms, which also may affect the performance of the device. Moisture transfer through this cover portion may be significant. However, it cannot be made too thick since it may be necessary (as discussed below, e.g., in regard to FIG. 25) to pierce the cover portion in order to expose the inlet port (or ports) through which the liquid sample that is to be measured is introduced. Therefore, the cover portion is preferably thin enough to be pierced easily, but is tough enough to withstand handling, while at the same time limiting moisture loss or intrusion. Examples of such materials include, but are not limited to, polypropylene, polystyrene, PET, polyethylene, polyesters, polyolefins such as cyclicolefin copolymers, COC, BCOP or LCP, PCTFE, PVC and multilayer materials such PCTFE, PVC, and CPC with polyesters, polyolefins or polyamides should also be appropriate. Other materials which may be used include polyethylene and polyesters such as Mylar® or SCO. A thickness of about 30 to 600 μm is preferred for most plastic materials. When the preferred polypropylene film is used, the thickness may be about 150 to 300 μm. The moisture transmission of the top layer should be about 0.007 to 0.01 g/m²-day, more generally 0.02 g/m²-day or below.

In various non-limiting embodiments of the presently claimed and disclosed inventive concept(s), the sample chamber (where present) may have a width in a range of 10 μm to 100 μm to 1000 μm to 5 mm to 10 mm, a depth in a range of 10 μm to 100 μm to 1000 μm to 5 mm, and a length in a range of 100 μm to 500 μm to 5 mm to 10 mm, the reaction chamber may have a width in a range of 10 μm to 100 μm to 1000 μm to 5 mm to 10 mm, a depth in a range of 10 μm to 100 μm to 1000 μm to 5 mm, and a length in a range of 100 μm to 500 μm to 5 mm to 10 mm, and the overflow chamber (where present) may have a width in a range of 10 μm to 100 μm to 1000 μm to 5 mm to 10 mm, a depth in a range of 10 μm to 100 μm to 1000 μm to 5 mm, and a length in a range of 100 μm to 500 μm to 5 mm to 10 mm. The microconduits between the inlet port, the chamber(s), and the air vent preferably have widths in the range of 10 μm to 100 μm to 500 μm to 1000 μm, and depths in the range of 10 μm to 100 μm to 500 μm to 1000 μm. The reaction chambers preferably contain one to twelve (or more) reagent substrates, typically ten, such as are described elsewhere herein.

The sample chamber (where present) and/or reaction chamber may contain microstructures disposed therein to reduce the capillary force exerted on the fluid sample as it moves through the chamber thereby evenly and uniformly distributing the sample across the chamber and displacing air therefrom.

While there are several ways in which the microconduits and chambers can be formed, such as injection molding, laser ablation, diamond milling or embossing, it is preferred to use injection molding in order to reduce the cost of the chips. Generally, a base portion (substrate) of the chip will be cut to create the microfluidic circuit of sample wells, overflow chamber(s), reaction chamber(s) and microconduit(s) and/or capillaries in either an upper surface or a lower surface of the base portion and then, after reagent substrate(s) have been placed in the wells as desired, a cover layer will be attached over or optionally under, the base to cover the microfluidic circuit and complete the chip. Holes for ports and vents may need to be drilled or otherwise positioned in the base portion and/or cover layer to access the microconduits.

In one version, the base portion (substrate) is a bottom of a container of the sample collection device and the microfluidic circuit is formed in the lower or outer surface of the bottom. In this version, the substrate can be made of an optically opaque or reflective material, and the cover layer can be made of an optically transparent material so that the reagent substrates can be optically read from a position beneath the container. Another important property of both the base portion and the cover layer are their optical clarity. When the response of a reagent to the presence or absence of an analyte in the sample is measured as a change in the color or in its intensity, or other wavelength, or in emission, absorbence, reflectance, or transmission of energy or wavelengths, the area of the cover layer adjacent to the measuring point should not interfere with the measurement. If the measurement is taken through the cover layer, then the cover layer should be optically transparent and the base portion optically opaque. In a preferred version, the cover layer is opaque or reflective (e.g., white) while the base of the device through which measurements are made is clear (transparent) or at least optically transparent. Exemplary optical transparent materials include glass, polystyrenes, polycarbonates, PET and the like. The base portion, the cover layer and the remainder of the sample collection device can be made out of the same or different materials so long as the reagent device within the sample collection device can be read by the analyzer 64.

The microfluidic devices (chips) used in the presently claimed and disclosed inventive concept(s) generally are intended to be disposable after a single use. Consequently, preferably they will be made of inexpensive materials to the extent possible, while being compatible with the reagents and the samples which are to be analyzed. In most instances, the chips will be made of plastics such as polycarbonate, polystyrene, polyacrylates, or polyurethane, alternatively, they can be made from silicates, glass, wax or metal. When moisture absorption by the plastic is not a substantial concern, the plastics preferably used may include, but are not limited to, ABS, acetals, acrylics, acrylonitrile, cellulose acetate, ethyl cellulose, alkylvinylalcohols, polyaryletherketones, polyetheretherketones, polyetherketones, melamine formaldehyde, phenolic formaldehyde, polyamides (e.g., nylon 6, nylon 66, nylon 12), polyamide-imide, polydicyclopentadiene, polyether-imides, polyethersulfones, polyimides, polyphenyleneoxides, polyphthalamide, methylmethacrylate, polyurethanes, polysulfones, polyethersulfones and vinyl formal. When moisture absorption is of concern, preferably the plastics used to make the chip include, but are not limited to: polystyrene, polypropylene, polybutadiene, polybutylene, epoxies, Teflon™, PET, PTFE and chloro-fluoroethylenes, polyvinylidene fluoride, PE-TFE, PE-CTFE, liquid crystal polymers, Mylar®, polyester, LDPE, HDPE, polymethylpentene, polyphenylene sulfide, polyolefins, PVC, and chlorinated PVC.

The microconduits of the microfluidic devices typically are hydrophilic, which is defined with respect to the contact angle formed at a solid surface by a liquid sample or reagent. Typically, a surface is considered hydrophilic if the contact angle is less than 90° and hydrophobic if the contact angle is greater than 90°. Preferably, plasma induced polymerization is carried out at the surface of the passageways. The microfluidic devices of the presently claimed and disclosed inventive concept(s) may also be made with other methods used to control the surface energy of the capillary walls, such as coating with hydrophilic or hydrophobic materials, grafting, or corona treatments. It is preferred that the surface energy of the capillary walls is adjusted, i.e. the degree of hydrophilicity or hydrophobicity, for use with the intended sample fluid, for example, to prevent deposits on the walls of a hydrophobic passageway or to assure that none of the liquid is left in a passageway. For most passageways in the presently claimed and disclosed inventive concept(s), the surface is generally hydrophilic since the liquid tends to wet the surface and the surface tension forces causes the liquid to flow in the passageway. For example, the surface energy of capillary passageways can be adjusted by known methods so that the contact angle of water is between 10° to 60° when the passageway is to contact whole blood or a contact angle of 25° to 80° when the passageway is to contact urine.

Movement of liquids through the capillary microconduits may be prevented or directed by capillary stops, which, as the name suggests, stop liquids from flowing through the capillary by a change in capillary forces. For example a more narrow capillary width can have a stronger stop strength than a less narrow capillary, thereby causing the fluid to move through the less narrow capillary in preference of movement through the more narrow capillary. Preferably in the presently claimed and disclosed inventive concept(s) flow is initiated by capillary forces driven by atmospheric pressure although in some embodiments flow may be initiated or reinitiated by other external forces such as automatic or manually driven pumps. Thus while not required in preferred embodiments of the presently claimed and disclosed inventive concept(s), it may be convenient in some instances to continue applying force while liquid flows through the capillary passageways in order to facilitate analysis. Absorbent materials, hydrostatic force, centrifugal force, and air or liquid vacuum and pressure can be used to overcome a stop. Flow can resume by capillary forces with or without the assistance of a pressure difference. Preferably, although the steps prevent liquid flow, they allow passage of air which allows air to be vented from the microfluidic system.

The hydrophilicity of capillaries, before a stop, at a stop, and after a stop has an impact on capillary stop strength. Using a stop that is wider and deeper than the capillary, referred to as a “capillary jump” can require accounting for the hydrophilic strength of surfaces before and after the “jump”. Furthermore, this hydrophilic strength of surfaces must be considered relative to the liquid being moved. If the change in dimensions between the capillary at the stop is not sufficient, then the liquid will not stop at the entrance to the wider area. It has been found that the liquid can eventually creep along the walls of the stop. Even with proper design of the shape, control of the degree of hydrophilicity is needed to control liquid movement even further so that stop is effective.

At a stop, a pressure difference may be required to be applied to overcome the effect of the stop. In general, the pressure difference needed is a function of the surface tension of the liquid, the cosine of its contact angle with the capillary and the change in dimensions of the capillary. That is, a liquid having a lower surface tension will require less force to overcome the stop than a liquid having a higher surface tension. A liquid which wets the walls of the hydrophilic capillary, i.e. it has a low contact angle, will require less force to overcome or “jump” the stop than a liquid which has a higher contact angle. The smaller the capillary, the greater the force which must be applied. This force can be generated by any means that allows a greater pressure before the stop than after the stop. In practice, a plunger pushing liquid into a port before the stop or pulling air out of a vent after the stop can provide the force to overcome the stop as effectively as applying a centrifugal force.

The microfluidic devices of the presently claimed and disclosed inventive concept(s) can take many forms as needed for the analytical procedures which measure the analyte of interest. As noted herein, the microfluidic devices typically employ a system of capillary passageways connecting wells or chambers containing dry or liquid reagents or conditioning materials. Analytical procedures may include prereacting the analyte to ready it for subsequent reactions, removing interfering components, mixing reagents, lysing cells, capturing biomolecules, carrying out enzymatic reactions, or incubating for binding events, staining, or deposition or others described herein or known in the art.

In general, it is desirable that samples are introduced at the inlet port over a very short time, preferably over one to 10 seconds, and more preferably over 0.5 sec to 2 sec. The passageways (microconduits) and chambers of a microfluidic chip are ordinarily filled with air. The small samples (e.g., 0.1 to 20 μL), should completely fill the microconduits and sample and reaction chambers to assure that accurate results are obtained from interaction of the samples with reagents. If the air is not purged completely from a chamber containing a reagent, only a partial response of the reagent may be obtained.

Since a liquid sample may be introduced in several ways, the actual shape of the opening in the inlet port may vary. The shape of the opening is not considered to be critical to the performance, since several shapes have been found to be satisfactory. For example, it may be merely a circular opening into which the sample is placed. Alternatively, the opening may be tapered to engage a corresponding shape in a pipette, capillary, or outlet which deposits the sample. Such ports may be sealed closed so that nothing can enter the microfluidic chip until the port is engaged by the device holding the sample fluid, such as a cup or pipette. Depending on the carrier type, the sample may be introduced by a positive pressure, as when a plunger is used to force the sample into the inlet port. Alternatively, the sample may be merely placed at the opening of the inlet port and capillary action used and atmospheric pressure to pull or push the sample into the microfluidic device. Excess sample is preferably not to be left on a surface however, since cross-contamination may occur. Also, in alternate embodiments, the sample may be placed at the opening of the inlet port and a vacuum used to pull the sample into the microfluidic chip. As has already been discussed, when the opening is small, sufficient capillary forces are created by the interaction of the passage walls and the surface tension of the liquid. Typically, biological samples contain water and the walls of the inlet port and associated passageways will be hydrophilic so that the sample will be drawn into the microfluidic chip even in the absence of added pressure. However, it should be noted that a negative pressure at the inlet port is not desirable, since it may pull liquid out of the inlet chamber. Means should be provided to prevent a negative pressure from being developed during the introduction of the sample. In the presently claimed and disclosed inventive concept(s) a vent to the atmosphere is provided behind the sample liquid for this purpose.

The sample inlet chamber (where present) may not be empty. It may contain reagents and/or filters. For example, the sample chamber may contain glass fibers for separating red blood cells from plasma so that they do not interfere with the analysis of plasma. Blood anti-coagulants may be included in the sample chamber.

As noted above, the microfluidic chips of the presently claimed and disclosed inventive concept(s) may comprise one or more overflow chambers so that, excess sample may be transferred thereto, in order to be sure that a sufficient amount of the sample liquid has been introduced into the reaction chamber for the intended analytical procedure. This is possible when the air vents and any liquid outlet passageways are provided with capillary stops so that the excess liquid is forced to flow into the overflow well. Where the sample is difficult to see easily, because of its color and/or small size, the overflow chamber may contain an indicator. By a change in color for example, when the sample enters the overflow chamber the indicator shows the person or machine carrying out the analysis that the microfluidic device has been filled. One such indicator reagent is the use of a buffer and a pH indicator dye such that when the indicator reagent is wet the pH causes the dye to change color from its dry state. Many such color transitions are known to those skilled in the art as well as reductive chemistries and electrochemical signals producing reaction.

Any one of the chambers of the microfluidic device may comprise microstructures which are used to assure purging of air from a microfluidic chamber and to uniformly contact liquid sample with a reagent or conditioning agent which has been disposed on a substrate in the chamber. Typically, the reagents will be liquids which have been coated on a porous support and dried. Distributing a liquid sample uniformly and at the same time purging air from the well can be done with various types of microstructures. Thus, they may also be useful in the sample inlet chambers discussed above.

For example, the microstructures may comprise an array of posts disposed in a reagent area so that the liquid sample must pass from the inlet port in a non-linear direction. The liquid is constantly forced to change direction as it passes through the array of posts. Air is purged from the reagent area as the sample liquid surges through the array of posts. Each of the posts may contain one or more wedge-shaped cutouts which facilitate the movement of the liquid as discussed in U.S. Pat. No. 6,296,126.

Other types of microstructures which are useful include three dimensional post shape with cross sectional shapes that can be circles, stars, triangles, squares, pentagons, octagons, hexagons, heptagons, ellipses, crosses or rectangles or combinations. Microstructures with two dimensional shapes such as a ramp leading up to reagents on plateaus may also be useful.

Microfluidic devices of the presently claimed and disclosed inventive concept(s) have many applications. Analyses may be carried out on samples of many fluids of biological origin which are fluids or have been fluidized including, but not limited to, blood, urine, bladder wash, saliva, sputum, spinal fluid, intestinal fluid, intraperitoneal fluid, food, blood, plasma, serum, cystic fluids, ascites, sweat, tears, feces, semen, nipple aspirates, and pus. Blood and urine are of particular interest. Also included are processed biological fluids such as milk, juices, wines, beer, and liquors. Fluids of non-biological origin or which may be contaminated, such as water, are also included. A sample of the fluid to be tested is deposited in the inlet port of the microfluidic device and subsequently into the reaction chamber thereof (via a sample chamber if present) to react with a reagent and to be analyzed. Biological samples analyzed herein may be obtained from any biological sample including humans or any other mammal, birds, fish, reptiles, amphibians, insects, crustaceans, marine animals, plants, fungi, and microorganisms. The reacted sample will be assayed for the analyte of interest, including for example a protein, a cell, a small organic molecule, or a metal. Examples of such proteins include, but are not limited to, albumin, HbAlc, protease, protease inhibitor, CRP, esterase and BNP. Cells which may be analyzed include E. coli, Pseudomonas sp., white blood cells, red blood cells, H. pylori, Streptococcus sp., Chlamydia and mononucleosis pathogens. Metals which may be detected include, but are not limited to, iron, manganese, sodium, potassium, lithium, calcium, and magnesium.

In many applications, it is desired to measure a color, light or wavelength emission developed by the reaction of reagents with the sample fluid and which may be measured or detected by analyzers known to those of ordinary skill in the art. It is also feasible to make electrical measurements of the sample, using electrodes positioned in the small wells in the chip. Examples of such analyses include electrochemical signal transducers based on amperometric, impedimetric, or potentimetric detection methods. Examples include the detection of oxidative and reductive chemistries and the detection of binding events.

It is contemplated that virtually any reagent used in the field of biological, chemical, or biochemical analyses could be used in the microfluidic devices of the presently claimed and disclosed inventive concept(s). Reagents undergo changes whereby the intensity, nature, frequency, or type of the signal generated is proportional to the concentration of the analyte measured in the clinical specimen. These reagents may contain indicator dyes, metals, enzymes, polymers, antibodies, electrochemically reactive ingredients and various other chemicals placed onto carriers (also referred to herein as reagent substrates). Carriers often used are papers, membranes or polymers with various sample uptake and transport properties. Liquid reagents, when used, are preferably isolated by barrier materials which prevent migration of water throughout the device, thus avoiding changes in the concentration through transpiration or evaporation and preventing moisture from reaching the dry reagents.

Any method of detecting and measuring an analyte in liquid sample can be used in the presently claimed and disclosed inventive concept(s). A variety of assays for detecting analytes are well known in the art and include, for example, enzyme inhibition assays, antibody stains, latex agglutination, and immunoassays, e.g., radioimmunoassay.

Immunoassays that determine the amount of protein in a biological sample typically involve the development of antibodies against the protein. The term “antibody” herein is used in the broadest sense and refers to, for example, intact monoclonal antibodies, polyclonal antibodies, multispecific antibodies (e.g., bispecific antibodies), and to antibody fragments that exhibit the desired biological activity (e.g., antigen-binding). The antibody can be of any type or class (e.g., IgG, IgE, IgM, IgD, and IgA) or sub-class (e.g., IgG1, IgG2, IgG3, IgG4, IgA1 and IgA2).

Immunoassays, including radioimmunoassay and enzyme-linked immunoassays, are useful in the methods of the presently claimed and disclosed inventive concept(s). A variety of immunoassay formats, including, for example, competitive and non-competitive immunoassay formats, antigen capture assays and two-antibody sandwich assays can be used in the methods of the invention (Self and Cook, Curr. Opin. Biotechnol. 7:60-65 (1996)).

Enzyme-linked immunosorbent assays (ELISAs) can be used in the presently claimed and disclosed inventive concept(s). In the case of an enzyme immunoassay, an enzyme is typically conjugated to the second antibody, generally by means of glutaraldehyde or periodate. As will be readily recognized, however, a wide variety of different conjugation techniques exist which are readily available to one skilled in the art.

In certain embodiments, the analytes are detected and measured using chemiluminescent detection. For example, in certain embodiments, analyte-specific antibodies are used to capture an analyte present in the biological sample and an antibody specific for the specific antibodies and labeled with an chemiluminescent label is used to detect the analyte present in the sample. Any chemiluminescent label and detection system can be used in the present methods. Chemiluminescent secondary antibodies can be obtained commercially from various sources. Methods of detecting chemiluminescent secondary antibodies are known in the art and are not discussed herein in detail.

Fluorescent detection also can be useful for detecting analytes in the presently claimed and disclosed inventive concept(s). Useful fluorochromes include, for example, DAPI, fluorescein, lanthanide metals, Hoechst 33258, R-phycocyanin, B-phycoerythrin, R-phycoerythrin, rhodamine, Texas red and lissamine. Fluorescent compounds, can be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic color visually detectable with a light microscope.

Radioimmunoassays (RIAs) can be useful in certain methods of the invention. Such assays are well known in the art. Radioimmunoassays can be performed, for example, with ¹²⁵I-labeled primary or secondary antibody.

In preferred embodiments, the microfluidic device of the presently claimed and disclosed inventive concept(s), comprises a disk, strip, or card for use in analysis of urine for components therein or aspects thereof, such as, but not limited to, leukocytes, nitrites, urobilinogen, proteins, albumin, creatinine, uristatin, calcium oxalate, myoglobin, pH, blood, specific gravity, ketone, bilirubin and glucose. The disk, strip, or card preferably contains a plurality of microfluidic units for analysis of multiple urine samples. The microfluidic units may be equally spaced in a radial or linear array and each is preferably configured to receive a separate sample distributed from the urine container.

Separation steps are possible in which an analyte is reacted with reagent in a first reaction chamber and then the reacted reagent or sample is directed to a second reaction chamber for further reaction. In addition a reagent can be re-suspended in a first reaction chamber and moved to a second reaction chamber for a reaction. An analyte or reagent can be trapped in a first or second chamber and a determination made of free versus bound reagent. The determination of a free versus bound reagent is particularly useful for multizone immunoassay and nucleic acid assays. There are various types of multizone immunoassays that could be adapted to this device. In the case of adaption of immunochromatography assays, reagents filters are placed into separate wells and do not have to be in physical contact as chromatographic forces are not in play. Immunoassays or DNA assay can be developed for detection of bacteria such as Gram negative species (e.g. E. coli, Enterobacter, Pseudomonas, Klebsiella) and Gram positive species (e.g. Staphylococcus aureus, Enterococcus). Immunoassays can be developed for complete panels of proteins and peptides such as albumin, hemoglobin, myoglobulin, α-1-microglobulin, immunoglobulins, enzymes, glycoproteins, protease inhibitors, drugs and cytokines (see, for examples: Greenquist in U.S. Pat. No. 4,806,311, Multizone analytical Element Having Labeled Reagent Concentration Zone, Feb. 21, 1989, Liotta in U.S. Pat. No. 4,446,232, Enzyme Immunoassay with Two-Zoned Device Having Bound Antigens, May 1, 1984).

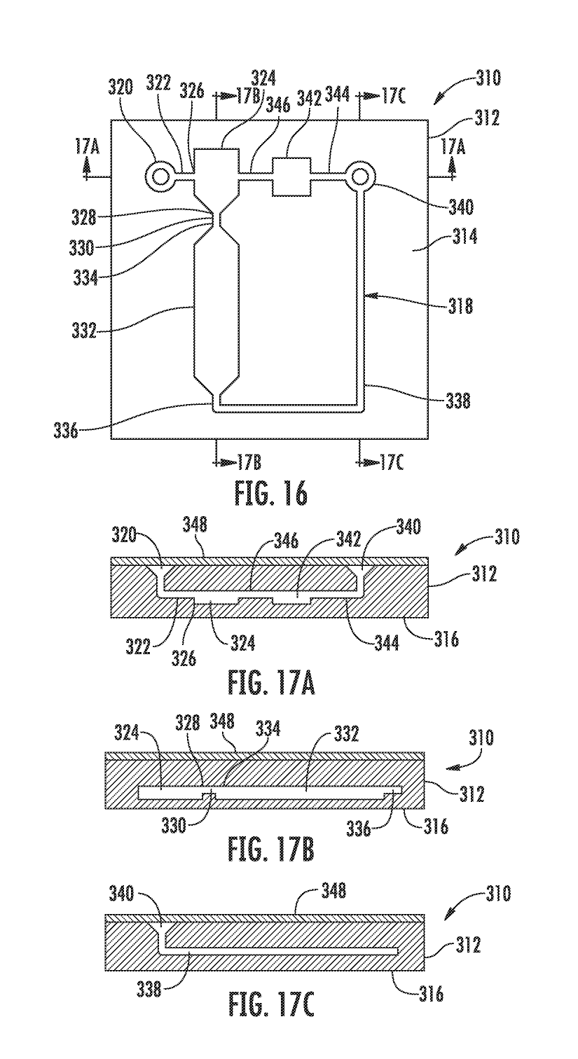

As described above, the sample chamber (when present, for example as shown in FIG. 16) which first receives the sample fluid should be filled completely and all the air ejected so that the desired amount of liquid is present in the sample chamber. However, if more than the desired amount of liquid is introduced, the excess should be removed. A passageway may therefore be provided between the sample chamber (where present) and an overflow chamber. However, since the sample chamber is connected to the reaction chamber of the microfluidic circuit, the liquid sample preferentially flows initially into the reaction chamber, rather than to the overflow chamber. It has been found that if a strong capillary stop is provided between the sample chamber and the overflow chamber, and an air vent is present between the overflow chamber and the reaction chamber, liquid first flows into the reaction chamber and only then does the excess liquid flow to the overflow chamber, where a visual means for detecting presence of the liquid may be provided. It may be desired that when the reaction chamber is full, excess liquid sample flows into the overflow chamber, rather than through an exit in the reaction chamber.

Referring now to FIGS. 14 and 15A-C, shown therein is a microfluidic device 210 which comprises a substrate 212 which is constructed of a material (such as described elsewhere herein) which is conventionally used for making microfluidic “chips.” The substrate 212 has an upper surface 214 and a lower surface 216. Formed into the substrate 212, by injection molding or etching, for example, is a microfluidic circuit 218 which comprises several ports, chambers and microconduits. More particularly, microfluidic circuit 218 comprises a sample inlet port 220, and a first sample microconduit 222 in fluid communication with a second sample microconduit 224. The sample inlet port 220 is in fluid communication with the first sample microconduit 222. The second sample microconduit 224 extends from the first sample microconduit 222 and fluidly connects to a reaction chamber 232 via a reaction chamber inlet 234.

The reaction chamber 232 has a reaction chamber outlet 236 which continues as a reaction chamber outlet microconduit 238 and is connected to an air vent 240 such that the sample inlet port 222, reaction chamber 232, and the air vent 240 are in fluid communication. Further, FIGS. 15A-C show the microfluidic device 210 constructed with a cover layer 248 which is disposed over the upper surface 214 of the substrate 212. The cover layer 248 is preferably constructed of a polymeric or metallic material and may be opaque, translucent, transparent, or reflective, depending on the particular circumstance under which the microfluidic device 210 is intended to be used. The cover layer 248 is preferably attached, bonded, or otherwise affixed to the upper surface 214, for example by chemical, heat, adhesive, ultrasonic, or physical bonding. Preferably an upper surface 250 of the cover layer 248 has an adhesive material thereon for use in a circumstance when it is desired to connect the microfluidic device 210 to a fluid sampling device such as a urine container in a manner such as discussed in further detail below.

Once a fluid sample (such as blood or urine or any other fluid which can be analyzed in accordance with the presently claimed and disclosed inventive concept(s)) enters the sample inlet port 220 it passes into the reaction chamber 232 via the first sample microconduit 222 and the second sample microconduit 224. The fluid sample flows unidirectionally in a direction such that the fluid flows into the reaction chamber 232. Therefore the microfluidic circuit 218 is designed, in one embodiment, such that each microconduit 222, 224 and 238 comprises a capillary stop which functions in accordance with a desired unidirectional flow of the fluid sample. In particular, in one embodiment, microconduit 238 may comprise a capillary stop which is stronger than the capillary stops of microconduits 222 and 224 which flow into the reaction chambers 232 such that fluid preferentially flows from the sample inlet port 220 into the reaction chamber 232 and fills the reaction chamber 232 completely before flowing into microconduit 238. Conversely, it is desired that air movement though the microfluidic circuit 218 ahead of the fluid flow be substantially unimpaired so that air within the microfluidic circuit 218 can be purged therefrom through the air vent 240 as the fluid sample flows therethrough from the sample inlet port 220 to the reaction chamber 232.

Referring now to FIGS. 16 and 17A-C, shown therein is a microfluidic device 310 which comprises a substrate 312 which is constructed of a material conventionally used for making microfluidic “chips” as described elsewhere herein. The substrate 312 has an upper surface 314 and a lower surface 316. Formed into the substrate 312, by injection molding or etching, for example, is a microfluidic circuit 318 which comprises several ports, chambers and microconduits which are in fluid communication with each other by virtue of a loop configuration. More particularly, microfluidic circuit 518 comprises a sample inlet port 320, a sample chamber inlet microconduit 322, a sample chamber 324, a sample chamber inlet 326, and a sample chamber outlet 328. The sample inlet port 320 is in fluid communication with the sample chamber 324 via the sample chamber inlet microconduit 322. The microfluidic circuit 318 further comprises a sample chamber outlet microconduit 330 which extends from the sample chamber outlet 328 and fluidly connects the sample chamber 324 to a reaction chamber 332 via a reaction chamber inlet 334.

The reaction chamber 332 has a reaction chamber outlet 336 which continues as a reaction chamber outlet microconduit 338 and is connected to an air vent 340 which is connected to an overflow chamber 342 via an overflow chamber-air vent microconduit 344 such that the reaction chamber 332, air vent 340 and overflow chamber 342 are in fluid communication. Finally, the overflow chamber 342 and sample chamber 324 are connected by a sample chamber-overflow chamber microconduit 346 such that the overflow chamber 342 and sample chamber 324 are in fluid communication. In view of the above, it can be seen that the microfluidic circuit 318 comprises a loop such that each chamber and microconduit is in fluid communication. Further, FIGS. 17A-C show the microfluidic device 310 constructed with a cover layer 348 which is disposed over the upper surface 314 of the substrate 312. The cover layer 348 is preferably constructed in a manner as discussed above and is preferably attached, bonded, or otherwise affixed to the upper surface 314, for example by chemical, heat, adhesive or physical bonding. Preferably an upper surface 350 of the cover layer 348 has an adhesive material thereon for use in a circumstance when it is desired to connect the microfluidic device 310 to a fluid sampling device such as a urine container in a manner such as discussed in further detail below.

Once a fluid sample (such as blood or urine or any other fluid which can be analyzed in accordance with the presently claimed and disclosed inventive concept(s)) enters the sample inlet port 320 and passes into the sample chamber 324 via the sample chamber inlet microconduit 322, the fluid sample in sample chamber 324 preferably flows unidirectionally in a direction such that the fluid initially flows into the reaction chamber 332 rather than into the overflow chamber 342. Therefore the microfluidic circuit 618 is designed, in one embodiment, such that each microconduit 322, 330, 338, 344 and 346 comprises a capillary stop which functions in accordance with a desired flow of the fluid sample. For example, microconduit 346, between the sample chamber 324 and the overflow chamber 342, may comprise a capillary stop which is stronger than the capillary stop of microconduit 330 between the sample chamber 324 and the reaction chamber 332 such that fluid preferentially flows from the sample chamber 324 into the reaction chamber 332 rather than into the overflow chamber 342. It is thus desired, in one embodiment, that the flow of sample fluid within microconduits 322, 330, 338 and 344 be generally unimpeded relative to the flow of fluid in microconduit 346 between sample chamber 324 and overflow chamber 342. Alternatively, it may be desired that the capillary stop of microconduit 346 is stronger than the capillary stop of microconduit 330 but is weaker than the capillary stop of microconduit 338 and 344 such that the flow of the fluid sample preferentially is in the direction of the overflow chamber 342 when the reaction chamber 332 is full such that flow of fluid sample out of the reaction chamber 332 through outlet 336 is minimized to reduce the dilution of “signal” which emanates from the reaction chamber 332, due to possible dilution of fluid sample within the reaction chamber 332. Conversely, it is desired that air movement though the microfluidic circuit 318 ahead of the fluid flow be substantially unimpaired so that air within the microfluidic circuit 318 can be purged therefrom through the air vent 340 as the fluid sample flows therethrough from the sample chamber 324 to the reaction chamber 332.

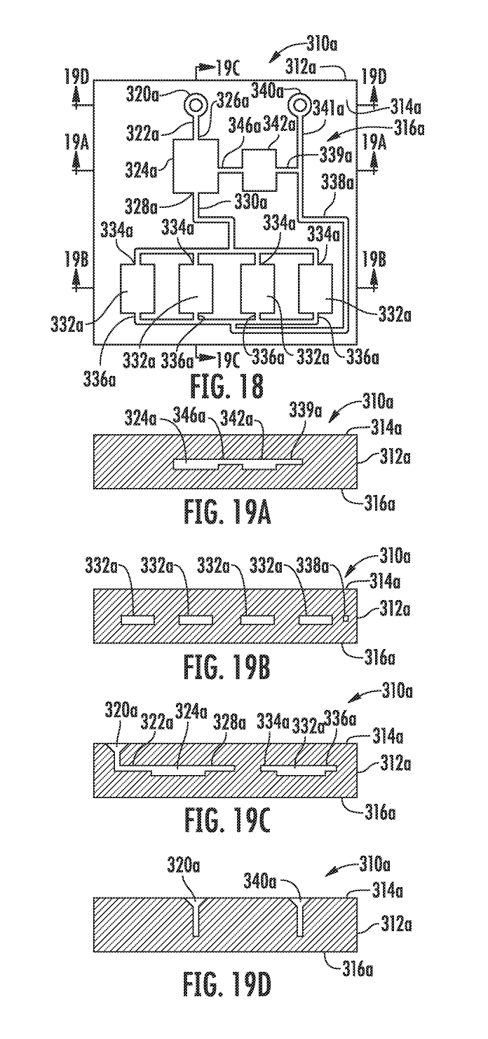

Shown in FIGS. 18 and 19A-D is an alternate embodiment of a microfluidic device of the presently claimed and disclosed inventive concept(s) and is designated therein by reference numeral 310a. The microfluidic device 310a is constructed in a manner similar to that described above for microfluidic device 310. The microfluidic device 310a comprises a substrate 312a which has an upper surface 314a and a lower surface 316a. Formed into the substrate 312a in a manner as discussed elsewhere herein is a microfluidic circuit comprising a microfluidic circuit 318a which comprises a sample inlet port 320a, a sample chamber inlet microconduit 322a, a sample chamber 324a, a sample chamber inlet 326a, and a sample chamber outlet 328a. The sample inlet port 320a is in fluid communication with the sample chamber 324a via the sample chamber inlet microconduit 322a. The microfluidic circuit 318a further comprises a sample chamber outlet microconduit 330a which extends from the sample chamber outlet 328a and fluidly connects the sample chamber 324a with each of a plurality of reaction chambers 332a via reaction chamber inlets 334a.

The reaction chambers 332a have reaction chamber outlets 336a which merge to continue as a reaction chamber outlet microconduit 338a which is connected to an air vent 340a via an air vent microconduit 341a and which is connected to an overflow chamber 342a via a reaction chamber-overflow chamber microconduit 339a such that the reaction chambers 332a, air vent 340a, and overflow chamber 342a are in fluid communication. Finally, the overflow chamber 342a and sample chamber 324a are connected by a sample chamber-overflow chamber microconduit 346a such that the overflow chamber 342a and sample chamber 324a are in fluid communication. In view of the above, it can be seen that the microfluidic circuit 318a comprises a loop wherein adjacent chambers and microconduits are in fluid communication with each other. Further, the microfluidic device 310a is optionally constructed with a cover layer (not shown) which may be constructed as shown above for cover layer 348 of microfluidic device 310, and which, may have, like cover layer 348, an adhesive upper surface for connecting to a sampling device in a manner consistent with the presently claimed and disclosed inventive concept(s).

As for microfluidic device 310, the fluid sample in microfluidic device 310a preferably flows in a direction such that fluid initially flows from sample chamber 324a into the reaction chambers 332a rather than into the overflow chamber 342a. Therefore the microfluidic circuit 318a is designed, in one embodiment, such that each microconduit 322a, 330a, 338a, 339a, 341a and 346a comprises a capillary stop which functions in accordance with the desired flow direction of the fluid sample. For example, microconduit 346a, between the sample chamber 324a and the overflow chamber 342a may comprise a capillary stop which is stronger than the capillary stop of microconduit 330a between the sample chamber 324a and the reaction chambers 332a such that fluid preferentially flows into the reaction chambers 332a rather than into the overflow chamber 342a. It is thus desired that the flow of sample fluid within microconduits 322a, 330a, 338a, 339a and 341a be generally unimpeded relative to the flow of fluid in microconduit 346a between sample chamber 324a and overflow chamber 342a. Alternatively, it may be desired that the capillary stop of microconduit 346a is stronger than the capillary stop of microconduit 330a but is weaker than the capillary stop of microconduit 338a and 339a such that the flow of the fluid sample preferentially is in the direction of the overflow chamber 342a when the reaction chambers 332a are full such that flow of fluid sample out of the reaction chambers 332a through outlets 336a is minimized to reduce the dilution of “signal” which emanates from the reaction chamber 332a due to possible dilution of fluid sample within the reaction chamber 332a. Conversely, it is desired that air movement though the microfluidic circuit 318a ahead of the fluid flow be substantially unimpaired so that air within the microfluidic circuit 318a can be purged therefrom through air vent 340a as the fluid sample flows therethrough from the sample chamber 324a to the reaction chambers 332a. Further, it is contemplated herein that any of the microfluidic devices described, enabled, or supported herein, such as those shown in FIGS. 14-19D can be constructed in configurations similar to those shown in FIG. 14 or 15A-C, or modifications thereof, wherein they are constructed without a sample chamber and/or an overflow chamber, and/or wherein they are constructed in a loop configuration (such as in FIG. 16) or in a non-loop (non-continuous) path (such as in FIG. 14). Further, for any of the microfluidic devices contemplated herein, all or some of the microconduits may comprise configurations designed to act as capillary stops. Further, the arrangements and geometries of the chambers, microconduits, and pathways of the microfluidic circuits of the invention may be different from those shown herein, which are intended to be exemplary only and non-limiting.

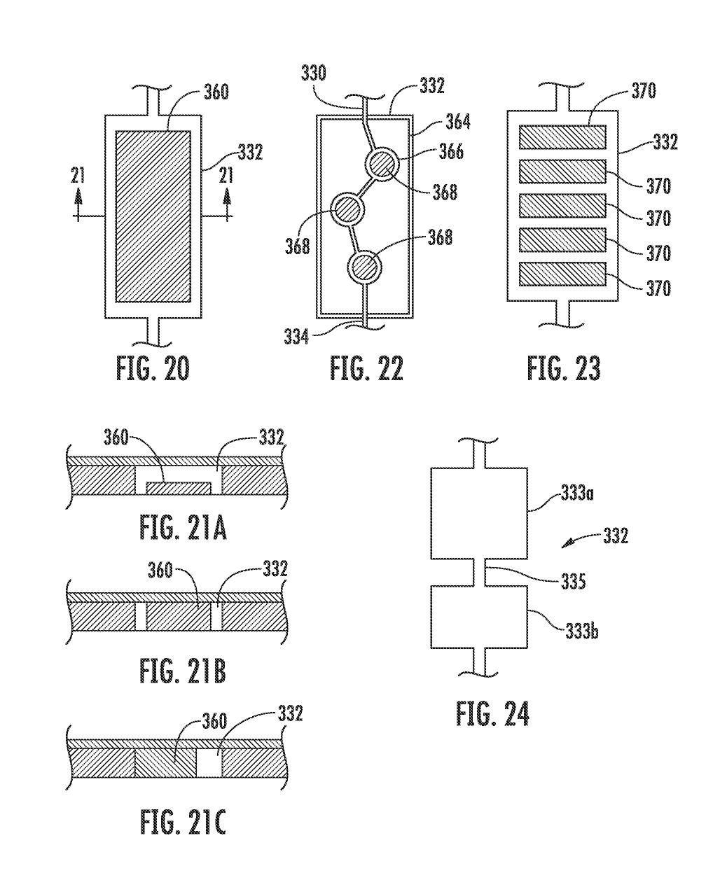

Shown in FIG. 20 is an embodiment of reaction chamber 332 (and may be considered to be representative of any reaction chamber of the presently claimed and disclosed inventive concept(s)) having a reagent substrate 360 disposed therein. Reagent substrate 360 preferably has, disposed thereon or therein, a dry or wet reagent for reacting with a component of the fluid sample for determining the presence and/or quantity of an analyte therein. Shown in FIGS. 21A-C are three configurations that the reagent substrate 360 can have within the reaction chamber 332. In FIG. 21A the reagent substrate 360 has dimensions such that it does not touch either the top or side walls of the reaction chamber 332. In FIG. 21B the reagent substrate 360 has dimensions such that an upper surface thereof touches the top of the reaction chamber 332 but does not touch the sidewalls thereof. In FIG. 21C the reagent substrate 360 has dimensions such that a side surface thereof touches a side wall of the reaction chamber 332 but does not touch the top of the reaction chamber 332. In an alternate embodiment (not shown) the reaction substrate 360 may substantially fill the reaction chamber 332.

Shown in FIG. 22 is an embodiment of a reaction chamber 332 (and may be considered to be representative of any reaction chamber of the presently claimed and disclosed inventive concept(s)) which comprises a microfluidic chip 364 which comprises a plurality of wells 366 which are connected in fluid communication by microconduits which are in alignment with the reaction chamber inlet 230 and the reaction chamber outlet 334. Reagent substrates 368 are disposed within the wells 366. FIG. 23 shows an embodiment of the reaction chamber 332 (and may be considered to be representative of any reaction chamber of the presently claimed and disclosed inventive concept(s)) which comprises a plurality of separate reagent substrates 370. The reagent substrates 370 may be positioned within the reaction chamber 332 in any one of the configurations shown in FIGS. 21A-C, or in any combination thereof or in any other suitable configuration. Shown in FIG. 24 is an embodiment of a reaction chamber 332 (and may be considered to be representative of any reaction chamber of the presently claimed and disclosed inventive concept(s)) and which comprises a separate first reaction chamber 333a and a separate second reaction chamber 333b which are connected by a microconduit 335. Each reaction chamber 333a and 333b may comprise reagent substrates or reaction wells as shown in FIGS. 20-23, for example. Other embodiments of the presently claimed and disclosed inventive concept(s) which have more than two interconnected reaction chambers, for example 3, 4, 5, 6, 7, 8, 9, 10, or more reaction chambers are contemplated herein.

Shown in FIG. 25 and designated therein by the general reference numeral 400 is an alternate embodiment of a microfluidic device of the presently claimed and disclosed inventive concept(s). The microfluidic device 400 comprises a substrate 402 comprising the same material used to construct the microfluidic devices described above, for example a clear plastic. The substrate 402 has a shape of a disk and is constructed with a plurality of microfluidic units 404 each comprising a plurality of chambers, microconduits and ports or vents which together comprise a microfluidic circuit 606. Each microfluidic unit 404 functions independently of each other microfluidic unit 404. The microfluidic units 404 are arranged radially in an array within the substrate 402. Eight microfluidic units 404 are shown in the microfluidic device 400, but it will be understood than any number of microfluidic units 404 may be formed within the substrate 402, for example, 1-60 or even more of such units 404 may be incorporated into substrate 402 if the substrate 402 is of sufficient size to accommodate them. The microfluidic units 404 as shown have microfluidic circuits which are similar to the microfluidic circuit 318 of microfluidic device 310 of FIG. 16. However, it will be understood that the microfluidic device 400 may be constructed using any of the microfluidic circuits contemplated or described herein which function in accordance with the presently claimed and disclosed inventive concept(s). The microfluidic device 400 is constructed so as to be adapted for placement on, attachment to, or engagement, with a bottom surface of a liquid collection container. The microfluidic device 400 may have a plurality of indexing means 408 such as alignment depressions, holes, posts, notches, or optically-readable symbols, or any other device known to those of ordinary skill in the alignment art for aligning the microfluidic device 400 on a lower surface of a liquid collection container, or other sample container. The microfluidic device 400 may also have an extension 410 extending therefrom for enabling the device 400 to be grasped by the user, or for aiding in moving the position of the device for example, by rotation, on the sampling device.

As described above for microfluidic devices described elsewhere herein, the microfluidic device 400 may have a cover layer (not shown) disposed thereon and which functions in the same manner as the cover layers described in regard thereto (such as for adhesion to the liquid container). The microfluidic device 400 is shown as having a disk shape, however it will be understood that the shapes of the microfluidic devices of the presently claimed and disclosed inventive concept(s), include but are not limited to, round, square, rectangular, irregular, oval, star, or any other geometric shape which allows the microfluidic circuits therein the function in accordance with the presently claimed and disclosed inventive concept(s).

For example, shown in FIG. 26 is another embodiment of the presently claimed and disclosed inventive concept(s) which comprises a microfluidic device designated by the general reference numeral 420. The microfluidic device 420 comprises a substrate 422 comprising the same material used to construct the microfluidic devices described elsewhere herein, for example a clear plastic. The substrate 422 has a rectangular shape and is constructed with a plurality of microfluidic units 424 each comprising a plurality of chambers, microconduits and ports or vents which together comprise a microfluidic circuit 426. Each microfluidic unit 424 functions independently of each other microfluidic unit 424. The microfluidic units 424 are arranged linearly in an array within the substrate 422. Six microfluidic units 424 are shown in the microfluidic device 420, but it will be understood than any number of microfluidic units 424 may be formed within the substrate 422, for example 1-60 or even more such units 424 may be incorporated into the substrate 422 if the substrate 422 is of sufficient size to accommodate them. The microfluidic units 424 as shown have microfluidic circuits which are similar to the microfluidic circuit 318 of microfluidic device 310 of FIG. 16. However, it will be understood that the microfluidic device 420 may be constructed with any of the microfluidic circuits contemplated or described herein which function in accordance with the presently claimed and disclosed inventive concept(s). The microfluidic device 420 is constructed so as to be adapted for placement on, attachment to, or engagement, with a side or bottom surface of a liquid collection container. The microfluidic device 420 may have a plurality of indexing means 428 such as alignment depressions, holes, posts, notches, or optically-readable symbols, or any other device known to those of ordinary skill in the art for aligning the microfluidic device 420 on a lower surface of a urine cup, or other sample container. The microfluidic device 420 may also have an extension 430 extending therefrom for enabling the device 420 to be grasped by the user, or for aiding in moving the position of the device for example, by pulling, pushing or drawing the sampling device.

As described above for microfluidic devices described elsewhere herein, the microfluidic device 420 may have a cover layer (not shown) disposed thereon and which functions in the same manner as the cover layers described in regard thereto (such as for adhesion to the liquid container). The microfluidic device 420 is shown as having a rectangular shape, however it will be understood that the shapes of the microfluidic devices of the presently claimed and disclosed inventive concept(s), include but are not limited to, round, square, rectangular, irregular, oval, star, or any other geometric, symmetric or asymmetric shape which allows the microfluidic circuit or circuits therein to function in accordance with the presently claimed and disclosed inventive concept(s). Further, any of the microfluidic devices described elsewhere herein may comprise an optically-readable or machine-readable symbol thereon, such as a bar code, as indicated by symbol 432 on microfluidic device 420.

As discussed elsewhere herein, the microfluidic device 210 (or any other of the microfluidic devices contemplated herein) of the presently claimed and disclosed inventive concept(s) are especially useful in the analysis of urine samples. FIGS. 27 and 28 show a sample collection device 500 including a container 502 which has a sidewall 504, a collection space 506 and a bottom 508. The bottom 508 has an upper surface 510 and a lower surface 512. A lid 514 is preferably disposed upon the container 502 to seal the inner space 506 and to provide a sealing device to seal the air capillary 520. The bottom 508 of container 502 has a first through hole which functions as a sample outlet 516 and a second through hole which functions as an air vent 518 which is connected in fluid communication to an air capillary 520 which is in fluid communication with the atmosphere when the lid 514 (or other sealing device such as a plastic film) is removed from the container 502 and which can remain in fluid communication with the atmosphere when the sample from the patient or subject is placed within the container. The lid 514 forms a removable sealing device covering a distal end 521 of the air capillary 520, however, other forms of removable sealing devices can be used such as tape. Removal of the sealing device permits air to flow through the air capillary 520 enabling the sample to enter the sample outlet 516 as discussed below. The sealing device can be removed by a patient, or hospital or laboratory personnel.

Connected to the lower surface 512 of the bottom 508 of the container 502 is a microfluidic device 522 which comprises a microfluidic circuit 524 constructed in accordance with the presently claimed and disclosed inventive concept(s). As shown in FIG. 28, the container 502 is used to collect a urine sample 526. Urine passes in direction 528 through the sample outlet 516 into the microfluidic circuit 524 and air is purged through the air vent 518 and the air capillary 520 via an air exit 530. After the urine sample 526 has reacted with reagents within the microfluidic device 522, the analyzer 64 can be used to detect and/or measure a signal emitted from the microfluidic device 522 as described elsewhere herein. Where used anywhere herein the term “air capillary” may also be referred to as an “air conduit” or “gas conduit” and may be a configuration other than a “capillary”, for example, it may have a width greater than its depth.

The distal end 521 of the air capillary 520 is positioned above the expected level of the sample 526 to be collected by the container 502. In the example shown in FIGS. 27 and 28, the distal end 521 of the air capillary is positioned adjacent to an upper end of the sidewall 504. However, the position of the distal end 521 can vary depending upon the size of the container 502 and the expected level of the sample 526. For example, the position of the distal end 521 may be above ½ the height of the sidewall 504.

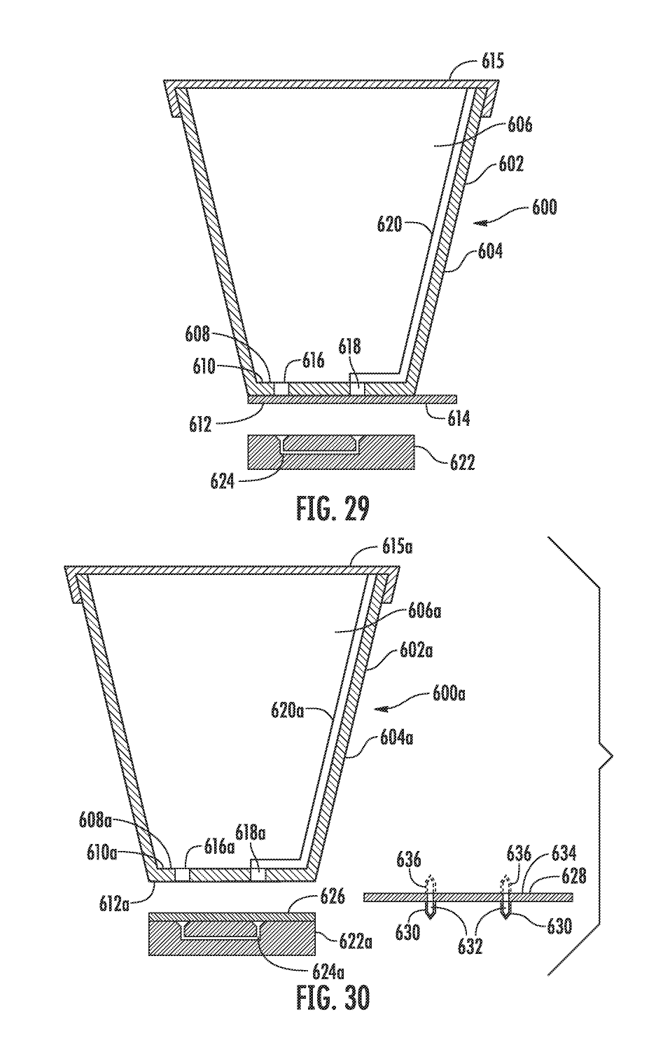

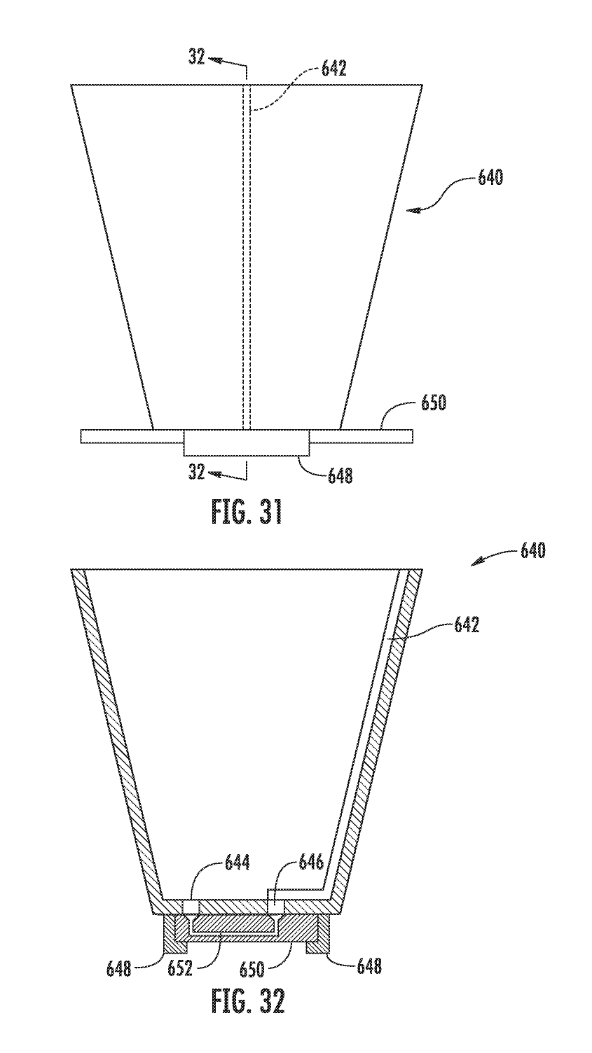

In the sample collection device 500 of FIGS. 27 and 28, the microfluidic device 522 is already connected to the container 502. However, in another embodiment as shown in FIGS. 29 and 30, sample collection devices are provided wherein the container and microfluidic device are not pre-attached. In FIG. 29, a sample collection device 600 comprises a container 602 which has a sidewall 604, a collection space 606 and a bottom 608. The bottom 608 has an upper surface 610 and a lower surface 612. The container 602 also preferably has a lid 615 (or film cover) which seals the collection space 606 thereof. A sealing layer 614 is disposed upon the lower surface 612 to cover a sample outlet 616, air vent 618, and air capillary 620 which comprise through holes in the bottom 608, until it is desired to use the container 602 at which time the sealing layer 614 is removed and the microfluidic device 622 having microfluidic circuit 624 is attached thereto. Alternatively, the microfluidic device 622 may have a removable cover, lid, or sealing layer (not shown) on an upper surface of the microfluidic device 622 which is removed prior to its application to the bottom 608 of the container 602.

In FIG. 30, a sample collection device 600a comprises a container 602a which has a wall 604a, a collection space 606a, and a bottom 608a. The bottom 608a has an upper surface 610a and a lower surface 612a. A lid 615a (or film cover) preferably covers the collection space 606a. A sample outlet 616a and an air vent 618a comprise through holes which pass through the bottom 608a. An air capillary 620a is connected to air vent 618a and is in fluid communication to the atmosphere when the lid 615a or another sealing means is removed. When it is desired to attach a microfluidic device 622a, having a microfluidic circuit 624a to the container 602a, a puncture device 628 having puncture spikes 630 each preferably with a through hole 632 is used to puncture a cover layer 626 upon an upper surface of the microfluidic device 622a to open an inlet port and air vent therein before the microfluidic device 622a is attached to the lower surface 612a of the container 602a in alignment with the sample outlet 616a and air vent 618a thereof. The puncture device 628 may be connectingly positioned between the lower surface 612a and the cover layer 626 of the microfluidic device 622a such that a sample in the container 602a and air in the device 622a flows through the through holes 632. In the event that the lower surface 612a of the container 602a has a cover layer or sealing layer thereon, it may be desirable for the puncture device 628 to have additional puncture spikes 636 (shown in phantom) on an upper surface 634 which are in fluid communication with puncture spikes 630 for the purpose of puncturing the cover layer on the lower surface 612a of the container 602a. Other means for perforating the cover layer 626 will be apparent to those of ordinary skill in the art. Alternatively, it may be desired to cause exposure of the microfluidic circuit 624a by simply removing the cover layer 626, rather than puncturing it, and attaching the uncovered microfluidic device 622a to the container 602a. Alternatively, the puncturing means may be incorporated in the bottom 608a of the container 602a wherein a separate puncture device 628 is not necessary.

FIGS. 31 and 32 show a sample container of the presently claimed and disclosed inventive concept(s) in an alternate embodiment and designated therein by general reference numeral 640. The container 640 is similar in construction to containers 502 and 602 in having an air capillary 642, a sample outlet 644 and air vent 646. Container 640 further comprises a microfluidic device track 648 which can support a microfluidic device 650 which has one or more microfluidic units 652 therein. The microfluidic device 650 can be any microfluidic device or microfluidic chip contemplated herein, each of which comprises at least one microfluidic unit having a microfluidic circuit. In this embodiment the microfluidic device 650 is inserted into the track 648 wherein the microfluidic unit 652 is aligned with the sample outlet 644 and air vent 646 of the container 640 so that the microfluidic unit 652 is in operational fluid communication with the container 640 for supplying a liquid sample to the microfluidic device 650. After a fluid sample is supplied to a first microfluidic unit 652 of the microfluidic device 650, the microfluidic device 6450 can be moved to a second operational position such that the sample outlet 644 and air vent 646 are aligned and in fluid communication with a second microfluidic unit 652. This process can be repeated until all or a portion of the microfluidic units 652 of the microfluidic device 650 are utilized by the user. The microfluidic device 650 can then be analyzed in situ within the track 648 or can be removed therefrom for analysis in accordance with the presently claimed and disclosed inventive concept(s).

The sample containers of the presently claimed and disclosed inventive concept(s) may comprise an outer sleeve which is integral with an inner sleeve or which is separable therefrom. The container may further comprise a handle. The air capillary in the collection container is preferably sealed until its use by the user or patient. For example, the air capillary may be sealed by a lid or cover over the entire cup or may be sealed by a sealing device such as a removable cover, film, plug, or stopper which only covers the exposed upper end of the air capillary.

It is also contemplated in accordance with the presently claimed and disclosed inventive concept(s), that a microfluidic device of the presently claimed and disclosed inventive concept(s) may be placed on a sidewall of a sample container rather than on a bottom surface. For example a sample outlet through hole and an air vent through hole may be located in the sidewall and the microfluidic device attached to an outer surface of the sidewall such that the sample inlet port of the microfluidic device is aligned with and in fluid communication with the sample outlet of the sample container and thus with the fluid sample therein, and such that the air vent of the microfluidic device is in alignment with and in fluid communication with the air vent and air capillary of the sample container. Alternatively, the microfluidic device may be attached to an inner surface of the sidewall or bottom surface of the sample container, as long as there are means for enabling a fluid sample to enter the microfluidic device, reading the reagent, and preferably means for venting air therefrom as well.

Although the presently claimed and disclosed inventive concept(s) and its advantages have been described in detail with reference to certain exemplary embodiments and implementations thereof, it should be understood that various changes, substitutions, alterations, modifications, and enhancements can be made to the presently claimed and disclosed inventive concept(s) described herein without departing from the spirit and scope of the presently claimed and disclosed inventive concept(s) as defined by the appended claims. Moreover, the scope of the presently claimed and disclosed inventive concept(s) is not intended to be limited to the particular embodiments of the processes, assemblies, items of manufacture, compositions of matter, means, methods and steps described in the specification. As one of ordinary skill in the art will readily appreciate from the disclosure of the presently claimed and disclosed inventive concept(s) many equivalent processes, assemblies, items of manufacture, compositions of matter, means, methods, or steps, presently existing or later to be developed that perform substantially the same function or achieve substantially the same result as the corresponding embodiments described herein may be utilized according to the presently claimed and disclosed inventive concept(s) disclosed herein. Accordingly, the appended claims are intended to include within their scope all such equivalent processes, assemblies, items of manufacture, compositions of matter, means, methods, or steps. Furthermore, each of the references, patents or publications cited herein is expressly incorporated by reference in its entirety.

What is claimed is:

1. A kit for a sample collection device, comprising:
a sample container having a sidewall, an inner space, a sample outlet and an air conduit; and
a microfluidic device attachable to the sample container and having at least one microfluidic circuit, wherein when the microfluidic device is attached to the sample container, the microfluidic circuit is placed in fluid communication with the sample outlet and the air conduit of the sample container, and the microfluidic circuit having a reaction chamber for receiving a fluid sample from the sample container.

2. The kit of claim 1 wherein:
the sample container comprises a sidewall, and a bottom having first and second through holes, with the first through hole forming the sample outlet, and the second through hole in fluid communication with the air conduit, the air conduit extending upwardly from the bottom of the sample container such that the air conduit is adapted to communicate with the air when a sample obtained from a patient is deposited into the sample container; and
the reaction chamber contains at least one substrate comprising a reagent for reacting with a component of the fluid sample.

3. The kit of claim 2, further comprising a removable sealing device covering a distal end of the air conduit.

4. The kit of claim 2 wherein the microfluidic device is attached to an outer surface of the bottom of the sample container in a position such that an inlet port of the at least one microfluidic circuit of the microfluidic device is aligned with the sample outlet of the sample container and the air vent of the microfluidic circuit of the microfluidic device is aligned with and in fluid communication with the air vent and air conduit of the sample container.

5. The kit of claim 1 wherein the microfluidic circuit of the microfluidic device comprises a sample chamber in fluid communication with the inlet port and with the reaction chamber.

6. The kit of claim 1 wherein the microfluidic circuit of the microfluidic device comprises an overflow chamber in fluid communication with the reaction chamber for containing an excess of the fluid sample.

7. The kit of claim 1 wherein the microfluidic device comprises a single microfluidic circuit.

8. The kit of claim 1 wherein the microfluidic device comprises a plurality of microfluidic circuits.

9. The kit of claim 1 wherein the reaction chamber of the microfluidic device comprises a plurality of reagent substrates for reacting with the fluid sample.

10. The kit of claim 1 wherein the reaction chamber of the microfluidic device comprises a plurality of separate compartments each of which is able to receive a portion of the fluid sample therefrom.

11. The kit of claim 1 wherein the reaction chamber of the microfluidic device comprises a reagent disposed upon a porous substrate.

12. The kit of claim 11 wherein the reagent disposed on the porous substrate is dry.

13. The kit of claim 11 wherein the reagent disposed on the porous substrate is a liquid.

14. A sample collection device comprising the sample container and microfluidic device of claim 1 in operative engagement.

15. A method of forming a sample collection device, comprising:
receiving the kit of claim 1;
attaching the microfluidic device of the kit to the sample container to form the sample collection device.

16. A kit for analyzing biological samples, comprising:
a sample collection device including:
a container defining a collection space adapted to collect and retain a sample directly from a patient, the container having a bottom; and
a reagent device located adjacent to the bottom of the container and in communication with the collection space to receive a portion of the sample; and
a portable reader comprising (1) a computer readable medium storing a code identifying at least one of a patient and a sample, (2) an analyzer and (3) a signal transceiver, the portable reader configured to mate with the container of the sample collection device for positioning the analyzer below the bottom of the container wherein when the portable reader is mated with the container and a read cycle is initiated the analyzer analyzes the reagent device to generate data indicative of the analysis of the reagent device and the signal transceiver outputs the code and the data indicative of the reagent device.

17. The kit of claim 16, wherein the portable reader comprises an actuator system adapted to communicate with the container for detecting and outputting data indicative of the entry of the sample into the container.

18. The kit of claim 17, further comprising at least one processor adapted to receive the data indicative of the entry of the sample into the container and automatically enable a read cycle for analyzing the reagent device.

19. The kit of claim 16, wherein the container also defines a reaction chamber adjacent to the bottom with the collection space and the reaction chamber having a volumetric ratio of at least 100 to 1, the container configured to establish fluid communication between the collection space and the reaction chamber.

20. The kit of claim 19, wherein the reagent device is positioned in the reaction chamber and extends across a portion of the bottom of the container to be optically readable from a position beneath the container.

21. A portable reader for automatically analyzing a sample collected from a patient with a sample collection device having a container defining a collection space of at least 75 mL and a reagent device positioned adjacent to a bottom of the container, comprising:
a computer readable medium initialized with a code identifying at least one of a patient and a sample;
an analyzer adapted to analyze the reagent device from a position beneath the bottom of the container; and
a signal transceiver adapted to output the code and data indicative of the analysis of the reagent device.

22. The portable reader of claim 21, further comprising a housing adapted to mate with the sample collection device to align the analyzer with the reagent device.

23. The portable reader of claim 21, wherein the portable reader further comprises an actuator system adapted to communicate with the sample collection device for detecting and outputting data indicative of the entry of the sample into a container of the sample collection device; and at least one processor adapted to receive the data indicative of the entry of the sample into the container and automatically enable a read cycle for analyzing the reagent device.

24. A method for analyzing a sample from a patient comprising the steps of:
initializing a portable reader with a code identifying at least one of a patient and a sample;
detecting, by the portable reader, the collection of the sample from the patient into a container of a sample collection device;
analyzing, in real-time, a reagent device of the sample collection device to generating data indicative of a reaction between the reagent device and the sample; and
transmitting the data and the code identifying at least one of the patient and the sample to a computer-based device external to the portable reader.

25. A method for collecting, analyzing and tabulating a sample from a patient, comprising the steps of:
initializing a portable reader with a code identifying at least one of a patient and a sample;
forming an assembled device by connecting the portable reader to a patient collection device;
providing the assembled device to a patient for collection of the sample whereby upon collection, the sample reacts with a reagent device of the sample collection device and data indicative of the reaction is automatically collected by the portable reader and tabulated into a medical database; and
obtaining the portable reader from the patient.

26. A kit for performing urinalysis, comprising:
a sample collection device including:
a container defining a collection space adapted to collect and retain urine directly from a patient; and
a reagent device in communication with the collection space to receive a portion of the urine;
a portable reader comprising an analyzer adapted to optically read the reagent device from a position below the container, the portable reader including a signal transceiver adapted to output (1) a unique code indicative of at least one of a patient and a sample, and (2) raw data indicative of the analysis of the reagent device; and
a host system adapted to execute a medical database and store the unique code and readable results into the medical database with the readable results indicative of the analysis of the reagent device.

27. The kit of claim 26, wherein the portable reader is adapted to mate with the sample collection device.

28. The kit of claim 26, wherein the portable reader comprises an actuator system adapted to communicate with the container for detecting and outputting data indicative of the entry of the sample into the container.

29. The kit of claim 28, further comprising at least one processor adapted to receive the data indicative of the entry of the sample into the container and to automatically enable a read cycle for analyzing the reagent device.

30. The kit of claim 26, further comprising a user device adapted to receive the raw data, convert the raw data into the readable results, and upload the readable results to the medical database of the host system.

31. The kit of claim 30, further comprising a base station adapted to provide computer executable instructions to the user device to facilitate the ability of the user device to convert the raw data into the readable results.

32. The kit of claim 26, wherein the host system receives the raw data and converts the raw data into the readable results.

33. A kit for analyzing and logging data indicative of the analysis of a biological sample in a container having a reagent device positioned adjacent to a bottom of the container, the kit comprising:
a portable reader comprising (1) a computer readable medium storing a code identifying at least one of a patient and a sample, (2) an analyzer and (3) a signal transceiver, the portable reader having an analyzer adapted to read the reagent device from a position beneath the bottom of the container wherein when a read cycle is initiated the analyzer analyzes the reagent device to generate data indicative of the analysis of the reagent device and the signal transceiver outputs the code and the data indicative of the reagent device; and
a host system executing a medical database receiving and storing the code and the data indicative of the analysis of the reagent device.

34. A sample collection device comprising:
a container having a bottom, and defining a collection space adapted to collect and retain a sample directly from a patient, the container also defining a reaction chamber adjacent to the bottom with the collection space and the reaction chamber having a volumetric ratio of at least 100 to 1, the container configured to establish fluid communication between the collection space and the reaction chamber; and
a reagent device positioned in the reaction chamber and extending across a portion of the bottom of the container to be optically readable from a position beneath the container.

35. The sample collection device of claim 34, wherein the container further comprises an air conduit and a sidewall with the air conduit fluidly connected to the reaction chamber and extending upwardly from the bottom of the container along the sidewall.
